(19) United States
(12) Patent Application Publication
(10) Pub. No.: US 2024/0424254 A1
(43) Pub. Date: Dec. 26, 2024

(54) DELIVERY SYSTEMS FOR CRYOABLATION DEVICE

(71) Applicant: Boston Scientific Scimed, Inc., Provinc, MN (US)

(72) Inventors: Kyle True, Minneapolis, MN (US); Timothy A. Ostroot, Cokato, MN (US); Cory Ross Stenberg, St. Michael, MN (US); Benjamin Wai-Man Chan, Edina, MN (US); Katrina Marie Daley, Corcoran, MN (US); David Cory Kirt, Minnetonka, MN (US); Zachary Nickle, Minneapolis, MN (US); Eric T. Gagner, Minnetonka, MN (US)

(21) Appl. No.: 18/671,727

(22) Filed: May 22, 2024

Related U.S. Application Data
(60) Provisional application No. 63/468,968, filed on May 25, 2023.

(51) Int. Cl. A61M 25/01; A61B 18/02

(52) U.S. Cl. CPC A61M 25/01; A61B 2018/0293

(57) ABSTRACT

In an embodiment, a cryoablation shaft is included having a working fluid circuit; a vacuum circuit; an insulated portion, wherein the vacuum circuit runs through the insulated portion; and an expansion chamber; a supply tube having a distal outlet in the expansion chamber, wherein fluid from the working fluid circuit travels distally down the cryoablation shaft through supply tube and expands in the expansion chamber; and a guidewire lumen including a metallic tube and a polymer sleeve configured to surround at least a portion of the metallic tube.

### RELATED APPLICATIONS

This application claims the benefit of U.S. Provisional Application No. 63/468,968, filed May 25, 2023, the content of which is herein incorporated by reference in its entirety.

### TECHNICAL FIELD

Embodiments herein relate to cryoablation systems and more particularly to delivery systems for cryoablation systems.

### BACKGROUND

During cryosurgery, a surgeon may deploy one or more cryoprobes to ablate a target area of a patient anatomy by freezing and thawing the tissue. In one example, a cryoprobe uses the Joule-Thomson effect to produce cooling or heating of the probe tip. In such cases, the expansion of a cryofluid in the cryoablation probe from a higher pressure to a lower pressure leads to cooling of the device tip to temperatures at or below those corresponding to cryoablation a tissue in the vicinity of the tip. Heat transfer between the expanded cryofluid and the outer walls of the cryoprobe leads to formation of an ice ball, in the tissue around the tip and consequent cryoablation of the tissue.

### SUMMARY OF THE INVENTION

In a first aspect, a cryoablation shaft includes a working fluid circuit, a vacuum circuit, an insulated portion, wherein the vacuum circuit runs through the insulated portion, and an expansion chamber extending distally to the insulated portion. The shaft further includes a guidewire lumen extending through a length of the cryoablation shaft, the guidewire lumen configured for insertion of a guidewire, the guidewire lumen can include a metallic tube and a polymer sleeve configured to surround at least a portion of the metallic tube. The shaft can further comprise a supply tube, the supply tube having a distal outlet in the expansion chamber, wherein fluid from the working fluid circuit travels distally down the cryoablation shaft through supply tube and expands in the expansion chamber, a return tube surrounding the supply tube, and an insulating shaft surrounding the return tube.

In a second aspect, in addition to one or more of the preceding or following aspects, or in the alternative to some aspects, the guidewire lumen can be concentrically located within the supply tube, the supply tube can be concentrically located within the return tube, and at least a portion of the return tube can be concentrically located within the insulating shaft.

In a third aspect, in addition to one or more of the preceding or following aspects, or in the alternative to some aspects, fluid from the working fluid circuit travels distally down the cryoablation shaft through an annular space defined between the return tube and the guidewire lumen.

In a fourth aspect, in addition to one or more of the preceding or following aspects, or in the alternative to some aspects, the metallic tube can include any of nitinol and stainless steel.

In a fifth aspect, in addition to one or more of the preceding or following aspects, or in the alternative to some aspects, the polymer sleeve can include any of polyether block amide and polyethylene terephthalate.

In a sixth aspect, in addition to one or more of the preceding or following aspects, or in the alternative to some aspects, at least a portion of the metallic tube includes slots, and the polymer sleeve can be configured to form a seal around the portion of the metallic tube can include the slots.

In a seventh aspect, in addition to one or more of the preceding or following aspects, or in the alternative to some aspects, the shaft further includes a distal tip configured to seal a distal end of the expansion chamber at a distal tip of the cryoablation shaft, wherein the distal tip includes metal.

In an eighth aspect, in addition to one or more of the preceding or following aspects, or in the alternative to some aspects, a distal end of the metallic tube can be joined to a proximal end of the distal tip using a metal-to-metal joining process.

In a ninth aspect, in addition to one or more of the preceding or following aspects, or in the alternative to some aspects, the distal tip defines a central channel, wherein the central channel can be configured to receive the guidewire, wherein a proximal end of the central channel of the distal tip can be joined to a distal end of the metallic tube.

In a tenth aspect, in addition to one or more of the preceding or following aspects, or in the alternative to some aspects, the shaft can include a first compressive wrap configured to seal the polymer sleeve to the metallic tube at a distal end of the polymer sleeve.

In an eleventh aspect, in addition to one or more of the preceding or following aspects, or in the alternative to some aspects, the shaft can include a second compressive wrap configured to seal the polymer sleeve to the metallic tube at a proximal end of the polymer sleeve.

In a twelfth aspect, in addition to one or more of the preceding or following aspects, or in the alternative to some aspects, the guidewire lumen may form a curve having a smallest radius of curvature of less than or equal to 30 mm.

In a thirteenth aspect, a guidewire lumen for a cryoablation shaft can include a metallic tube, wherein at least a portion of the metallic tube includes slots, a polymer sleeve, wherein the polymer sleeve can be configured to form a seal around the portion of the metallic tube can include the slots, and a distal tip. The guidewire lumen can be configured to extend through a length of the cryoablation shaft, the guidewire lumen can be configured for insertion of a guidewire, and the metallic tube can be configured to be joined to the distal tip at a distal end of the cryoablation shaft using a metal to metal joining processes.

In a fourteenth aspect, in addition to one or more of the preceding or following aspects, or in the alternative to some aspects, the guidewire lumen can be concentrically located within a supply tube of the cryoablation shaft, the supply tube can be concentrically located within a return tube of the cryoablation shaft, and at least a portion of the return tube can be concentrically located within an insulating shaft of the cryoablation shaft.

In a fifteenth aspect, in addition to one or more of the preceding or following aspects, or in the alternative to some aspects, the metallic tube can include any of nitinol and stainless steel.

In a sixteenth aspect, in addition to one or more of the preceding or following aspects, or in the alternative to some aspects, the polymer sleeve can include any of polyether block amide and polyethylene terephthalate.

In a seventeenth aspect, in addition to one or more of the preceding or following aspects, or in the alternative to some aspects, the guidewire lumen further can include a compressive wrap configured to seal the polymer sleeve to the metallic tube.

In an eighteenth aspect, in addition to one or more of the preceding or following aspects, or in the alternative to some aspects, the guidewire lumen may form a curve having a smallest radius of curvature of less than or equal to 30 mm.

In a nineteenth aspect, a cryoablation shaft can include a working fluid circuit, a vacuum circuit, an insulated portion, wherein the vacuum circuit runs through the insulated portion, and an expansion chamber, a guidewire lumen extending through a length of the cryoablation shaft, the guidewire lumen configured for insertion of a guidewire. The guidewire lumen can include a metallic tube wherein at least a portion of the metallic tube includes slots, and a polymer sleeve configured to surround at least a portion of the metallic tube, wherein the polymer sleeve can be configured to form a seal around the portion of the metallic tube that includes the slots. The shaft can further include a supply tube, the supply tube having a distal outlet in the expansion chamber, wherein fluid from the working fluid circuit travels distally down the cryoablation shaft through supply tube and expands in the expansion chamber, a return tube surrounding the supply tube, and an insulating shaft surrounding the return tube, wherein the guidewire lumen can be concentrically located within the supply tube, wherein the supply tube can be concentrically located within the return tube, wherein the return tube can be concentrically located within the insulating shaft.

In a twentieth aspect, in addition to one or more of the preceding or following aspects, or in the alternative to some aspects, the shaft further can include a distal tip configured to seal a distal end of the expansion chamber at a distal tip of the cryoablation shaft, wherein the distal tip includes metal.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cryoablation system in accordance with various embodiments herein.

FIG. 2 is a schematic view of portions of a cryoablation system in accordance with various embodiments herein.

FIG. 3 is a schematic view of a portion of a cryoablation shaft shown in accordance with various embodiments herein.

FIG. 4 is a cross sectional view of the shaft of FIG. 3 taken along section 4-4 in FIG. 3 in accordance with various embodiments herein.

FIG. 5 is a cross-sectional view of the shaft of FIG. 3 taken along section 5-5 in FIG. 3 in accordance with various embodiments herein.

FIG. 6 is a schematic view of the biliary system in accordance with various embodiments herein.

FIG. 7 is a schematic view of the growth of an ice ball generated by a cryoablation system in accordance with various embodiments herein.

FIG. 8 is a schematic view of a portion of a cryoablation shaft in accordance with various embodiments herein.

FIG. 9 is a schematic view of a portion of a cryoablation shaft in accordance with various embodiments herein.

FIG. 10 is a schematic view of a portion of a cryoablation shaft in accordance with various embodiments herein.

FIG. 11 is a radial cross-sectional view of a cryoablation shaft in accordance with various embodiments herein.

FIG. 12 is a radial cross-sectional view of a cryoablation shaft in accordance with various embodiments herein.

FIG. 13 is a radial cross-sectional view of a cryoablation shaft in accordance with various embodiments herein.

FIG. 14 is a cross-sectional view of a guidewire lumen and distal tip in accordance with various embodiments herein.

FIG. 15 is a cross-sectional view of a cryoablation shaft including the guidewire lumen and distal tip of FIG. 14, in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Some cryoablation systems may be useful for ablating lesions in the biliary system or other difficult to access portions of the human anatomy. In such cases, the cryoprobes may have to navigate tortuous passageways. Cryoprobes with rigid shafts may not be suitable for such applications.

The present disclosure is directed towards a cryoablation system having a flexible shaft. The shaft may be sufficiently flexible to access particular portions of the human anatomy, such as the biliary system, while simultaneously maintaining adequate burst strength and thermal insulation to ensure patient safety.

In various embodiments, it is desirable for the cryoablation system to include a guidewire. A guidewire as defined herein is a flexible, medical wire inserted into the body to guide a larger instrument, such as a catheter, to a target location. Including a guidewire in the cryoablation system can enable the flexible shaft to access portions of the patient's anatomy (e.g., the biliary system) with increased accuracy and decreased damage. A guidewire may run through a guidewire lumen that extends either part way or all the way though the length of the shaft. A guidewire can be added to the cryoablation system in either a monorail or an over-the-wire configuration. In alternate embodiments, the cryoablation system may be introduced to a patient using a sheath rather than a guidewire. Advantageous guidewire lumen constructions are described herein.

The concepts described herein can be applied in the context of the cryoablation systems described in US Published Patent Application US2021/00045793, titled “Dual Stage Cryocooler,” and US Published Patent Application US2021/00045794, titled “Flexible Cryoprobe,” both filed Aug. 14, 2020, and both incorporated herein by reference in their entireties.

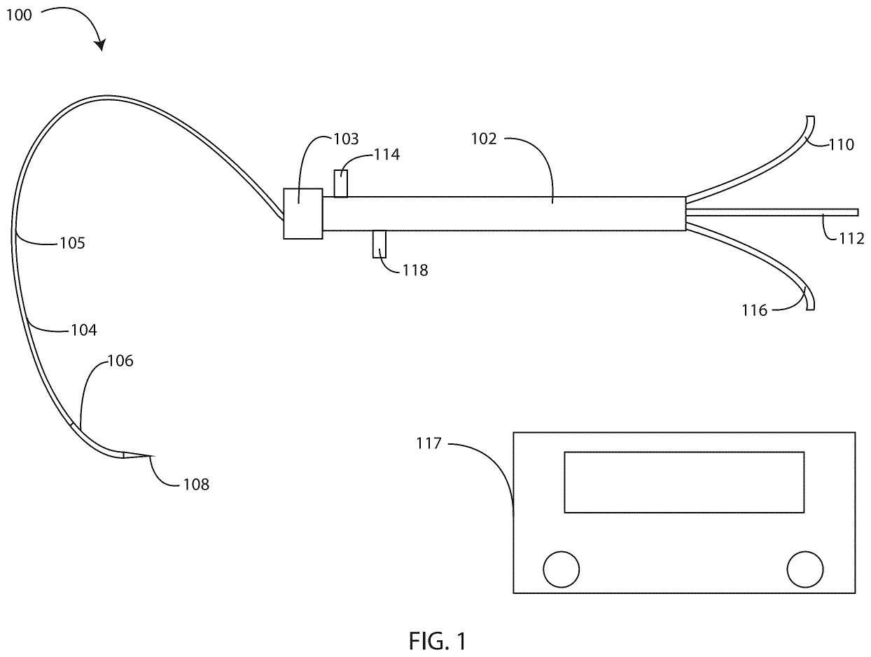

Referring now to FIG. 1, a schematic view of a cryoablation system is shown in accordance with various embodiments herein. In various embodiments, the cryoablation system can include a handle 102 and a shaft 104. In various embodiments, the shaft 104 is insertable into the handle 102 and can be securely attached to the handle with shaft-handle connector 103. In various embodiments, the shaft 104 and the shaft-handle connector 103 of a cryoablation system 100 can form a catheter assembly. In some embodiments, the catheter assembly includes the components of the cryoablation system that are to be replaced each time a cryoablation procedure is performed. In some aspects, the cryoablation system 100 may include a working fluid source 110, a pre-cooler fluid source 112, and vacuum source 114 which are each connectible to the cryoablation system 100.

The three sources correspond to three independent circuits in the cryoablation system 100: pre-cooler, working fluid, and active vacuum. In some embodiments, the working fluid source 110 and pre-cooler fluid source 112 connect to the base of the handle 102 of the cryoablation system 100 and vacuum source 114 connects near the distal end of the handle, adjacent to the shaft-handle connector 103. The cryoablation system may further include a pre-cooler gas exhaust 116 and a working gas exhaust 118 connecting to the handle 102. In various embodiments, the shaft-handle connector 103 functions as a manifold to ensure each of the flow circuits remain isolated from one another.

In some embodiments, the cryoablation system 100 includes a console 117. The console may be used to control the system and may be in electrical and fluid communication with the handle and cryoablation assembly. In some embodiments, the working fluid source 110, pre-cooler fluid source 112, vacuum source 114 may all be connectable to a console 117 of the cryoablation system 100 using conduits. In some embodiments, the pre-cooler gas exhaust 116, working gas exhaust 118, or both can connect to a conduit which carries the exhaust back to the console 117 or other location in the procedure room where the exhaust is vented to the ambient environment at an appropriate location. It should be noted that various sources and exhausts may be placed in position and in any suitable configuration along the handle 102, and that the arrangement of FIG. 1 is just one example of a suitable configuration.

An example of specifications and functions of each of these circuits is provided in the following paragraph. However, it should be noted that the particular fluids and pressure values are meant for exemplary purposes and other configurations are possible.

In an embodiment, the pre-cooler circuit can contain 24.1 mega Pascals (MPa) pressurized Argon. The precooler circuit can cool the incoming stream of working fluid and can operate in the handle. In an embodiment, the working fluid circuit can contain 12.4 MPa pressurized Argon and/or 12.4 MPa pressurized Helium. The working fluid circuit generates and/or thaws ice balls. The working fluid circuit can operate in the handle, the insulated portion or insulated zone of the shaft, and the expansion chamber of the shaft. In an embodiment, the active vacuum can hold a vacuum of less than or equal to 6.67 Pascals (Pa). The active vacuum can insulate the shaft. The active vacuum can operate in the handle and the insulated zone of the shaft.

In various embodiments, the working fluid circuit runs through both the handle 102 and the shaft 104 of the cryoablation system 100 and carries the fluid which both generates and thaws the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. During the ablation (freeze cycle), 12.4 MPa argon is circulated through the probe to generate the ice ball in the patient's body surrounding the expansion chamber 106. The working fluid can be any suitable cooling fluid (e.g., nitrogen, air, argon, krypton, xenon, N2O, CO2, CF4). In some embodiments, the pressure of the high-pressure stream of the working fluid can be greater than or equal to 6.9 MPa, 8.3 MPa, 9.7 MPa, 11.0 MPa, 12.4 MPa, 17.2 MPa, 27.6 MPa, or 41.4 MPa. In some embodiments, the pressure of the high-pressure stream of the working fluid can be less than or equal to 55.2 MPa, 34.5 MPa, 20.7 MPa, 18.6 MPa, 16.5 MPa, 14.5 MPa, or 12.4 MPa. In some embodiments, the pressure of the high-pressure stream of the working fluid can fall within a range of 6.9 MPa to 41.4 MPa, or 8.3 MPa to 27.6 MPa, or 9.7 MPa to 16.5 MPa, or 11.0 MPa to 14.5 MPa, or can be about 12.4 MPa. Accordingly, in the embodiments where the working fluid is a cooling fluid, the temperature of the working fluid at the expansion chamber 106 can be about 190 Kelvin. In some embodiments, the temperature of the working fluid can be less than or equal to 250 Kelvin, 200 Kelvin, 150 Kelvin, or 100 Kelvin, or can be an amount falling within a range between any of the foregoing.

In various embodiments, the pre-cooler circuit is fully contained within the handle 102. In various embodiments, the pre-cooler circuit is located in a console 117 of the system. In various embodiments, the pre-cooler circuit is located in a part of the catheter just proximal to the handle. In various embodiments, the pre-cooler circuit is located in a part of the catheter just distal to the handle. The pre-cooler circuit operates using argon or any other suitable cooling fluid in various embodiments. In some embodiments, the high-pressure stream of the pre-cooler fluid may be at a pressure greater than the pressure of the high-pressure stream of the working fluid. The pre-cooler fluid may, for instance, be supplied at pressures greater than about 13.8 MPa. In some embodiments, the pressure of the pre-cooler fluid can be greater than or equal to 10.3 MPa, 13.8 MPa, 17.2 MPa, 20.7 MPa, or 24.1 MPa. In some embodiments, the pressure of the pre-cooler fluid can be less than or equal to 31.0 MPa, 29.3 MPa, 25.9 MPa, or 24.1 MPa. In some embodiments, the pressure of the pre-cooler fluid can fall within a range of 10.3 MPa to 31.0 MPa, or 13.8 MPa to 29.3 MPa, or 17.2 MPa to 27.6 MPa, or 20.7 MPa to 25.9 MPa, or can be about 24.1 MPa.

In some embodiments, the outer surface of the shaft 104 may be thermally insulated from the inner surface of the shaft. In various embodiments, the vacuum circuit or vacuum chamber runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft. In alternative embodiments, shaft insulation can be obtained by containing a non-circulating fluid or gas within an insulating shaft.

The shaft 104 can be of any suitable length capable of reaching the target anatomy in the subject. In some embodiments, the shaft length can be greater than or equal to 20 cm, 38 cm, 55 cm, 72 cm, or 90 cm. In some embodiments, the shaft length can be less than or equal to 150 cm, 135 cm, 120 cm, 105 cm, or 90 cm. In some embodiments, the shaft length can fall within a range of 20 cm to 150 cm, or 38 cm to 135 cm, or 55 cm to 120 cm, or 72 cm to 105 cm, or can be about 90 cm.

In various embodiments, certain portions of the shaft 104 may be flexible. In an embodiment, the entire length of the shaft may be flexible. For instance, the shaft may be bendable about its lengthwise axis. In some such embodiments, the shaft may have a shaft diameter configured such that the shaft may be sufficiently flexible to form a curve having a desired radius of curvature. For instance, the shaft may be sufficiently flexible, such that the shaft may form a curve having the smallest radius of curvature of less than or equal to 30 mm, 20 mm, 10 mm, or 5 mm.

In various embodiments, shaft 104 may include an insulated zone 105 and an expansion chamber 106. The insulated zone 105 defines the portion of shaft 104 that is insulated by the vacuum chamber. The expansion chamber 106 defines the portion of the shaft 104 that is not insulated by the vacuum and where the ice ball is generated. In various embodiments, flexible shaft carries high pressure working fluid from the handle 102 to the expansion chamber 106, where it undergoes a Joule-Thompson expansion and corresponding temperature change. The working fluid exits down the flexible shaft, through the handle, before venting to the atmosphere from the console, or into the handle and venting from the handle.

The distal end of the shaft may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

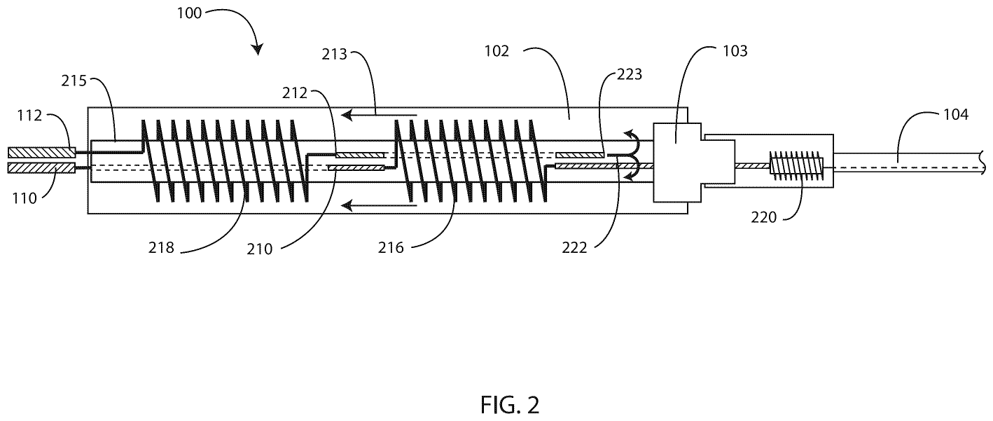

### Handle with Pre-Cooler Circuit (FIG. 2)

Referring now to FIG. 2, a schematic view of portions of a cryoablation system is shown in accordance with various embodiments herein. In some aspects, the cryoablation system 100 may include a working fluid source 110 connecting to a working fluid circuit and a pre-cooler fluid source 112 connecting to a pre-cooler fluid circuit. The working fluid circuit may include a working fluid supply conduit 210 for carrying a high-pressure stream of the working fluid from the working fluid source 110 to the distal end of the shaft 104 (not shown in this view). The working fluid circuit may also include a working fluid return conduit (not shown in this view) for carrying a low-pressure stream of the working fluid from the distal end of the shaft back to the base of the handle 102.

The pre-cooler fluid circuit may include a pre-cooler supply circuit 212, which terminates at pre-cooler Joule-Thomson orifice 223 and carries a high-pressure stream of a pre-cooler fluid from the pre-cooler fluid source 112 to the pre-cooler fluid expansion region 222 in the handle 102. The pre-cooler fluid circuit may also include a pre-cooler return conduit (marked by arrows 213). The pre-cooler return conduit may be configured to carry the pre-cooler fluid away from the pre-cooler fluid expansion region 222 back to the base of the handle 102. The pre-cooler return conduit may be housed along with the pre-cooler supply circuit 212 and extend back to a control console and gas manifold.

In various embodiments, the pre-cooler fluid circuit may facilitate heat exchange between the working fluid and the pre-cooler fluid. For instance, the pre-cooler fluid circuit can be used to precool the high-pressure stream of the working fluid in embodiments where the working fluid cools upon expansion to cryogenically ablate tissue surrounding the distal operating tip 108. In various embodiments, the working fluid supply conduit 210 may include a first heat exchanger 216. The first heat exchanger 216 may facilitate heat exchange between the high-pressure stream of the working fluid in the working fluid supply conduit 210 and the low-pressure stream of the pre-cooler fluid in the pre-cooler return conduit.

In various embodiments, the pre-cooler supply conduit 212 may include a second heat exchanger 218 that permits heat exchange between the high-pressure stream of the pre-cooler fluid and the low-pressure stream of the pre-cooler fluid (e.g., recuperative heat exchange). In various embodiments aspects, the pre-cooler fluid may also be a cooling fluid. In such embodiments, recuperative heat exchange between the high-pressure stream of the pre-cooler fluid and the low-pressure stream of the pre-cooler fluid may remove heat from the high-pressure stream of the pre-cooler fluid. Accordingly, the second heat exchanger 218 may facilitate precooling the high-pressure stream of the pre-cooler fluid.

In various embodiments, the high-pressure stream of the pre-cooler fluid leaving the second heat exchanger 218 continues to flow through the pre-cooler supply conduit 212 to the pre-cooler fluid expansion region 222. In the pre-cooler fluid expansion region, which is fully contained in handle 102, the pre-cooler supply conduit 212 terminates in a Joule-Thomson orifice. The high-pressure stream of the pre-cooler fluid may undergo expansion at or downstream of the Joule-Thomson orifice in the pre-cooler fluid expansion region 222. The rapid drop in pressure causes a corresponding drop in temperature. The pre-cooler fluid expansion region 222 may be in fluid communication with the pre-cooler return conduit to carry the expanded low-pressure stream of the pre-cooler fluid (e.g., to vent to atmosphere, if the pre-cooler fluid circuit is an open circuit, or back to a pre-cooler fluid source if the pre-cooler fluid circuit is a closed circuit). After expansion at the Joule-Thomson orifice, the chilled pre-cooler fluid passes back through handle 102, in the annular space between the core tube 215 and the outer surface of the handle 102. As the pre-cooler fluid passes through the pre-cooler return conduit, it cools the working fluid at the first heat exchanger 216.

The working fluid circuit 210 may also include a third heat exchanger 220 in the shaft 104 of the cryoablation system that is configured for heat exchange (e.g., recuperative heat exchange) between the high-pressure stream of the working fluid in the working fluid supply circuit 210 and the low-pressure stream of the working fluid returning through the shaft 104 (not shown in this view).

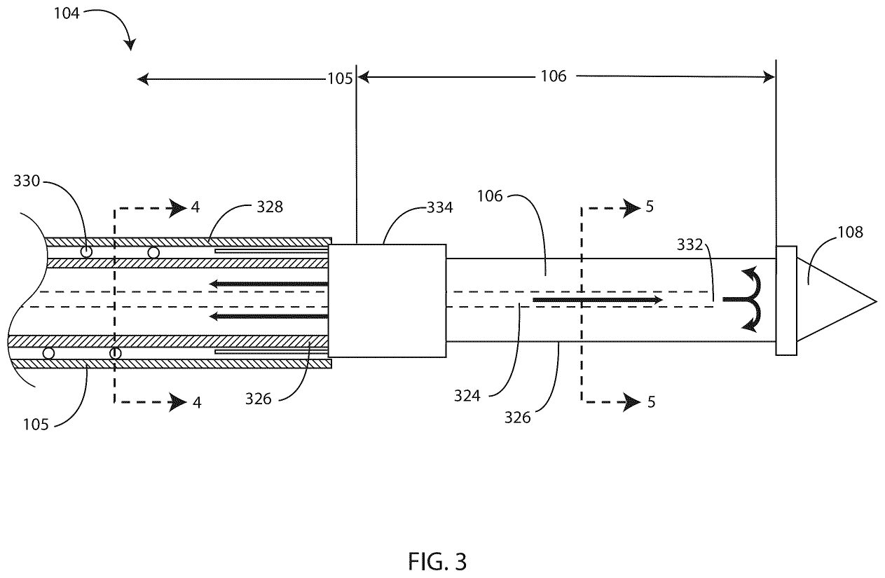

### Distal Tip and Expansion Chamber Details (FIG. 3)

Referring now to FIG. 3, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 which is located within a return tube 326, which is located within an insulating shaft 328. The concentric-shaft construction is designed to isolate the working fluid circuit 210 and vacuum chamber 336 from each other.

In various embodiments, after exiting the handle 102, the high-pressure flow of the working fluid travels down the supply tube 324. When the working fluid reaches the working fluid expansion chamber 106, the supply tube 324 terminates in a Joule-Thomson orifice 332 or distal outlet 332. The high-pressure stream of the working fluid may undergo expansion at or downstream of the Joule-Thomson orifice 332 in expansion chamber 106. The rapid drop in pressure causes a corresponding drop in temperature. Heat transfer between the expanded working and the outer walls of expansion chamber 106 leads to formation of an ice ball in the tissue around the tip 108 resulting in cryoablation of the tissue.

The expansion chamber 106 may be in fluid communication with the working fluid return conduit (defined by the annular space between the supply tube 324 and the inner surface of the return tube 326 of the expansion chamber) to carry the expanded low pressure stream of the working fluid (e.g., to vent to atmosphere, if the working fluid circuit is an open circuit, or back to a working fluid source if the working fluid circuit is a closed circuit). As the working fluid passes through the working fluid return conduit, it cools the working fluid input stream at the third heat exchanger 220 (FIG. 2).

In various embodiments, the working fluid is a cooling fluid and a cooling gas (e.g., nitrogen, air, argon, krypton, xenon, N₂O, CO₂, CF₄). In such cases, the high-pressure stream of the working fluid may be at a pressure such that expansion via the

Joule-Thomson orifice 332 may result in the working fluid cooling to temperatures for cryogenically ablating tissue surrounding the expansion chamber 106. In certain aspects, the pressure of the high-pressure stream of the working fluid upstream of the Joule-Thomson orifice 332 can be between about 6.9 MPa and about 13.8 MPa (e.g., about 12.4 MPa). Accordingly, in the embodiments where the working fluid is a cooling fluid, the temperature of the working fluid after expansion from the Joule-Thomson orifice 332 can be greater than or equal to 150, 160, 170, 180, 190, or 200 Kelvin, or can be an amount falling within a range between any of the foregoing.

Cryoablation system 100 can be designed such that the outermost surface of the shaft does not cause thermal damage to non-target structures. In various embodiments, Ice ball formation is limited to the expansion chamber 106 of the shaft 104, which can also be referred to as the active region of the device. Selective ice ball formation is achieved by pulling a vacuum through the insulated zone 105 of shaft 104. In various embodiments, the cryoablation system 100 may be configured for establishing vacuum communication between the shaft 104 and vacuum source 114.

Referring back to FIG. 1, cryoablation system 100 may be configured to connect to a vacuum source 114 at handle 102. In various embodiments, the vacuum source 114 is configured to pull vacuum along the length of the insulated zone 105 of shaft 104. In an embodiment, vacuum is pulled between the outer diameter of the return tube 326 and the inner diameter of the insulating shaft 328 throughout the insulated zone 105 of shaft 104.

In various embodiments, the vacuum source 114 is configured to pull a vacuum within at least a portion of the handle 102. Such a configuration can insulate the handle 102 and protect the cryoablation system operator from cryogenic exhaust gases. In some embodiments the vacuum source 114 is connected to the handle 102 and the shaft 104 is in fluid communication with the handle 102 such that pulling a vacuum in the handle can also evacuate the space between the supply tube 324 and return tube 326. In other embodiments, the vacuum source 114 is connected directly to the shaft 104, for example, with the use of a t-fitting along the length of the shaft 104.

To provide for thermal insulation along the insulated zone 105 of shaft 104, the wall of the flexible shaft is a double wall (a return tube surrounded by an insulating shaft) with a small gap between the return tube 326 and the insulating shaft 328. By pulling a vacuum between the return tube and the insulating shaft, convective heat transfer is prevented, so that the temperature of the working fluid does not ablate or cause uncontrolled apoptosis/necrosis to healthy non-target patient tissue along the insulated zone of the shaft. Adequate thermal insulation is obtained by actively pumping out the air in the gap and maintaining a vacuum of about 0.05 torr. However, other vacuum pressures may be appropriate depending on the configuration of the cryoablation system. In some embodiments, a supporting filament 330 is wrapped around the outer diameter of the return tube 326. One option for the filament material is a polymer such as polyether ether ketone (PEEK). The filament may prevent direct contact between the return tube outer surface and insulating shaft inner surface. Filament 330 minimizes thermal conduction between the inner and insulating shafts. Other alternatives may be used in place of the filament 330 such as an extruded tubing/co-extrusion shape or other features placed onto the shaft.

In some embodiments, the shaft may not include a filament. In such embodiments the return tube 326 and the insulating shaft 328 are selected to have material properties that are sufficient to minimize thermal conduction between the inner shaft and the insulating shaft.

A joint 334 is present at the junction of the insulated zone 105 and the expansion chamber 106. This joint is capable of sealing the vacuum layer.

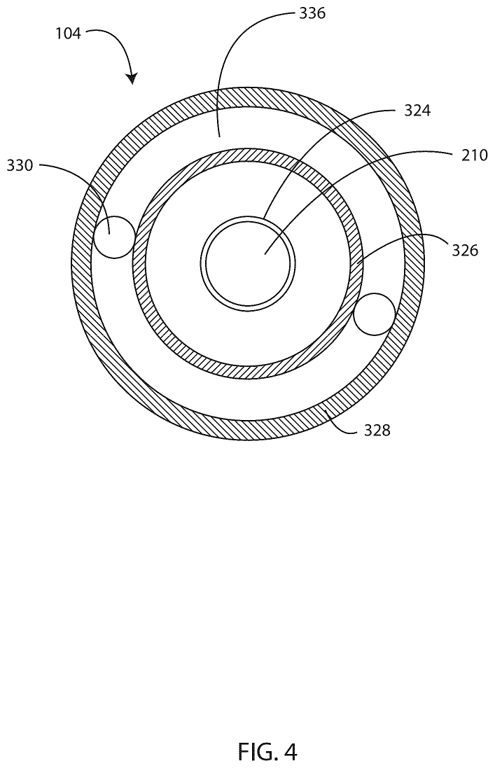

### Flexible Shaft Cross-Section, Dimensions and Materials (FIG. 4)

Referring now to FIG. 4, a cross sectional view of the shaft of FIG. 3 taken along section 4-4 is shown in accordance with various embodiments herein. In various embodiments, the insulated zone 105 of shaft 104 includes a supply tube 324 concentrically located within a return tube 326, which is concentrically located within an insulating shaft 328. The insulated zone 105 can include a shaft portion of the vacuum chamber 336 and an insulated portion of the working gas circuit 210. In various embodiments, the vacuum chamber 336 surrounds and is isolated from the insulated portion of the working gas circuit 210.

In various embodiments, after exiting the handle 102, the high-pressure flow of the working fluid travels distally down the insulated zone of the shaft through supply tube 324. After cooling and expansion in the expansion chamber 106, the working fluid travels proximally back through the insulated zone 105 of the shaft 104 in the annular space between the supply tube 324 and return tube 326.

In various embodiments, the materials and dimensions of each of the layers of the shaft 104 may be selected to provide sufficient degree of flexibility for the shaft to be bendable about its longitudinal axis at the working temperatures of the device.

In various embodiments, the supply tube 324, which also may be referred to as a capillary tube herein, is constructed from any suitable material or materials such as flexible metals, polymers, composites, or the like. In an embodiment, the supply tube 324 is constructed from Nitinol (NiTi), stainless steel, or the like.

In some embodiments, the inner diameter of the supply tube 324 can be greater than or equal to 0.30 mm, 0.35 mm, 0.40 mm, or 0.45 mm. In some embodiments, the inner diameter of the supply tube 324 can be less than or equal to 0.60 mm, 0.55 mm, 0.50 mm, or 0.45 mm. In some embodiments, the diameter of the supply tube 324 can fall within a range of 0.30 mm to 0.60 mm, or 0.35 mm to 0.55 mm, or 0.40 mm to 0.50 mm, or can be about 0.45 mm.

In some embodiments, the outer diameter of the supply tube 324 can be greater than or equal to 0.38 mm, 0.43 mm, 0.48 mm, 0.53 mm, or 0.58 mm. In some embodiments, the outer diameter can be less than or equal to 0.78 mm, 0.73 mm, 0.68 mm, 0.63 mm, or 0.58 mm. In some embodiments, the outer diameter can fall within a range of 0.38 mm to 0.78 mm, or 0.43 mm to 0.73 mm, or 0.48 mm to 0.68 mm, or 0.53 mm to 0.63 mm, or can be about 0.58 mm.

In some embodiments, the thickness of the supply tube 324 can be greater than or equal to 0.10 mm, 0.11 mm, 0.12 mm, 0.14 mm, or 0.15 mm. In some embodiments, the thickness of the supply tube 324 can be less than or equal to 0.20 mm, 0.19 mm, 0.18 mm, 0.16 mm, or 0.15 mm. In some embodiments, the thickness of the supply tube 324 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the return tube 326 is constructed from any suitable material or materials such as flexible metals, polymers, or the like. In various embodiments, the return tube 326 can be made of polyimide, fluorinated ethylene propylene (FEP), Teflon, or the like. In an embodiment, the return tube 326 is formed from a polyimide material as it is highly impermeable to gases at a wide range of temperatures and can thus contain the working fluid inside and hold vacuum on the outside. In a particular example, the return tube 326 is made of a braid-reinforced polyimide tube to enhance gas impermeability, burst strength, and flexibility. In some embodiments, the return tube 326 is formed from a single layer of material. In some embodiments, the return tube 326 can be formed from two or more layers of material selected to optimize the performance of the shaft 104. The layers of material can be bonded together using any suitable technique or techniques such as adhesives, reflow processes, or the like.

In some embodiments, the outer diameter of the return tube 326 can be greater than or equal to 1 mm, 1.1 mm, 1.2 mm, 1.3 mm, or 1.4 mm. In some embodiments, the outer diameter of the return tube 326 can be less than or equal to 1.8 mm, 1.7 mm, 1.6 mm, 1.5 mm, or 1.4 mm. In some embodiments, the outer diameter of the return tube 326 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the inner diameter of the return tube 326 can be greater than or equal to 0.9 mm, 1.0 mm, 1.1 mm, 1.2 mm, or 1.3 mm. In some embodiments, the inner diameter of the return tube 326 can be less than or equal to 1.7 mm, 1.6 mm, 1.5 mm, 1.4 mm, or 1.3 mm. In some embodiments, the inner diameter of the return tube 326 can fall within a range of 0.9 mm to 1.7 mm, or 1.0 mm to 1.6 mm, or 1.1 mm to 1.5 mm, or 1.2 mm to 1.4 mm, or can be about 1.3 mm.

In some embodiments, the thickness of the return tube 326 can be greater than or equal to 0.10 mm, 0.11 mm, 0.12 mm, 0.14 mm, or 0.15 mm. In some embodiments, the thickness of the return tube 326 can be less than or equal to 0.20 mm, 0.19 mm, 0.18 mm, 0.16 mm, or 0.15 mm. In some embodiments, the thickness of the return tube 326 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the insulating shaft 328 is constructed from any suitable material or materials such as flexible metals, polymers, or the like. In various embodiments, the insulating shaft 328 is made of polyimide, fluorinated ethylene propylene (FEP), Teflon, or the like. In a particular embodiment, the insulating shaft 328 may include polytetrafluoroethylene (PTFE), and/or one or more polyether block amides (known under the tradename Pebax®, hereinafter “Pebax”).

In some embodiments, the insulating shaft 328 is formed from a single layer of material. In some embodiments, the insulating shaft 328 can be formed from two or more layers of material selected to optimize the performance of the shaft 104. The layers of material can be bonded together using any suitable technique or techniques such as adhesives, reflow processes, or the like.

In an embodiment, the insulating shaft may be formed using a braid-reinforced polyimide tube skim-coated with a Pebax outer layer. Such a tri-layer construction enables the deep vacuum to be maintained between the return tube and the insulating shaft without causing the insulating shaft 328 to collapse onto the return tube 326.

In some embodiments, the outer diameter of the insulating shaft 328 can be greater than or equal to 1.2 mm, 1.4 mm, 1.5 mm, 1.6 mm, or 1.8 mm. In some embodiments, the outer diameter of the insulating shaft can be less than or equal to 2.2 mm, 2.1 mm, 2.0 mm, 1.9 mm, or 1.8 mm. In some embodiments, the outer diameter of the insulating shaft can fall within a range of 1.3 mm to 2.3 mm, or 1.4 mm to 2.1 mm, or 1.5 mm to 2.0 mm, or 1.6 mm to 1.9 mm, or can be about 1.8 mm.

In some embodiments, the inner diameter of the insulating shaft 328 can be greater than or equal to 1.0 mm, 1.2 mm, 1.3 mm, 1.4 mm, or 1.6 mm. In some embodiments, the inner diameter of the insulating shaft 328 can be less than or equal to 2.2 mm, 2.0 mm, 1.9 mm, 1.8 mm, or 1.6 mm. In some embodiments, the inner diameter of the insulating shaft 328 can fall within a range of 1.0 mm to 2.2 mm, or 1.2 mm to 2.0 mm, or 1.3 mm to 1.9 mm, or 1.4 mm to 1.8 mm, or can be about 1.6 mm.

In some embodiments, the thickness of the insulating shaft 328 can be greater than or equal to 0.10 mm, 0.11 mm, 0.12 mm, 0.14 mm, or 0.15 mm. In some embodiments, the thickness of the insulating shaft 328 can be less than or equal to 0.20 mm, 0.19 mm, 0.18 mm, 0.16 mm, or 0.15 mm. In some embodiments, the thickness of the insulating shaft 328 can fall within a range of 0.10 mm to 0.20 mm, or 0.11 mm to 0.19 mm, or 0.12 mm to 0.18 mm, or 0.14 mm to 0.16 mm, or can be about 0.15 mm.

In some embodiments, a PEEK filament 330 is wound around the return tube 326. PEEK filament 330 may have pitch that is greater than or equal to 0.5 mm, 1.0 mm, 1.5 mm, or 2.0 mm, or can be an amount falling within a range between any of the foregoing. Alternatively, the filament may be a plurality of discrete pieces attached along the return tube 326. The PEEK filament 330 prevents direct contact between the return tube 326 and the insulating shaft 328, maintaining their coaxial alignment. In some embodiments, an adhesive (e.g., Loctite) is applied on the filament at the end of the return tube 326 and the insulating shaft 328 to attach the PEEK filament 330. In various embodiments, the PEEK filament wrap is configured to minimize or prevent conductive heat transfer from the return tube to the insulating shaft. In alternative embodiments, other insulating polymers may be used as a substitute for the PEEK filament such as expanded PTFE (ePTFE), nylon, or the like.

In some embodiments, the diameter of the PEEK filament 330 can be greater than or equal to 0.002 mm, 0.004 mm, or 0.005 mm. In some embodiments, the diameter of the PEEK filament 330 can be less than or equal to 0.007 mm, 0.006 mm, or 0.005 mm. In some embodiments, the diameter of the PEEK filament 330 can fall within a range of 0.002 mm to 0.007 mm, or 0.004 mm to 0.006 mm, or can be about 0.005 mm.

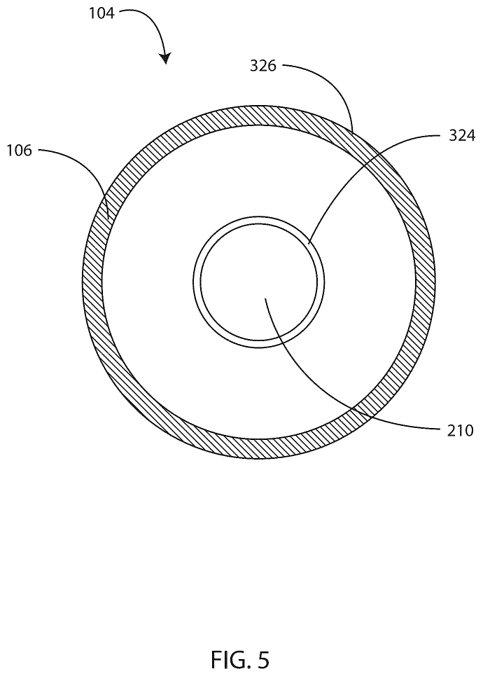

### Shaft in the Expansion Chamber (FIG. 5)

Referring now to FIG. 5, a cross-sectional view of the shaft of FIG. 3 taken along section 5-5 is shown in accordance with various embodiments herein. The cross-sectional view of FIG. 5 depicts expansion chamber 106 of the shaft 104. In various embodiments, the expansion chamber 106 is distal to the insulated zone 105 along the shaft 104. The expansion chamber 106 can include an expansion portion of the working fluid circuit 210.

In various embodiments, after exiting the handle 102, the high-pressure flow of the working fluid travels down the supply tube 324. After cooling and expansion in expansion chamber 106, the working fluid travels back down through the expansion chamber 106 in the annular space between the supply tube 324 and the outer wall of the expansion chamber. In various embodiments, the expansion chamber 106 is configured to maximize the heat transfer between the working gas and the patient's tissue through the optimization of parameters such as wall thickness, materials, and the like.

In various embodiments, the expansion chamber 106 is constructed from any suitable material or materials such as flexible metals, polymers, or the like. In various embodiments, the expansion chamber 106 is made of polyimide, fluorinated ethylene propylene (FEP), Teflon, or the like. In some embodiments, the expansion chamber 106 includes a continuation of the return tube 326 of the insulated zone 105 of the shaft 104. Alternatively, the expansion chamber is a separate component from the return tube 326 that can be joined to the shaft 104 using any suitable joint and/or fitting, such as reflow processes, glue joints, solder joints, or any other suitable mechanical joining process capable of withstanding cryogenic pressures and temperatures.

In some embodiments, the expansion chamber 106 is formed from a single layer of material. In some embodiments, the expansion chamber 106 is formed from two or more layers of material. The layers of material can be bonded together using any suitable technique or techniques such as adhesives, reflow processes, or the like.

In some embodiments, the outer diameter of the expansion chamber 106 can be greater than or equal to 1.3 mm, 1.4 mm, 1.5 mm, 1.6 mm, or 1.7 mm. In some embodiments, the outer diameter of the expansion chamber 106 can be less than or equal to 2.1 mm, 2.0 mm, 1.9 mm, 1.8 mm, or 1.7 mm. In some embodiments, the outer diameter of the expansion chamber 106 can fall within a range of 1.3 mm to 2.1 mm, or 1.4 mm to 2.0 mm, or 1.5 mm to 1.9 mm, or 1.6 mm to 1.8 mm, or can be about 1.7 mm.

In some embodiments, the inner diameter of the expansion chamber 106 can be greater than or equal to 1.0 mm, 1.1 mm, 1.2 mm, 1.3 mm, or 1.4 mm. In some embodiments, the inner diameter of the expansion chamber 106 can be less than or equal to 1.8 mm, 1.7 mm, 1.6 mm, 1.5 mm, or 1.4 mm. In some embodiments, the inner diameter of the expansion chamber 106 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the thickness of the wall of expansion chamber 106 can be greater than or equal to 0.20 mm, 0.22 mm, 0.25 mm, 0.28 mm, or 0.30 mm. In some embodiments, the thickness of the wall of the expansion chamber 106 can be less than or equal to 0.40 mm, 0.38 mm, 0.35 mm, 0.32 mm, or 0.30 mm. In some embodiments, the thickness of the wall of the expansion chamber 106 can fall within a range of 0.20 mm to 0.40 mm, or 0.22 mm to 0.38 mm, or 0.25 mm to 0.35 mm, or 0.28 mm to 0.32 mm, or can be about 0.30 mm.

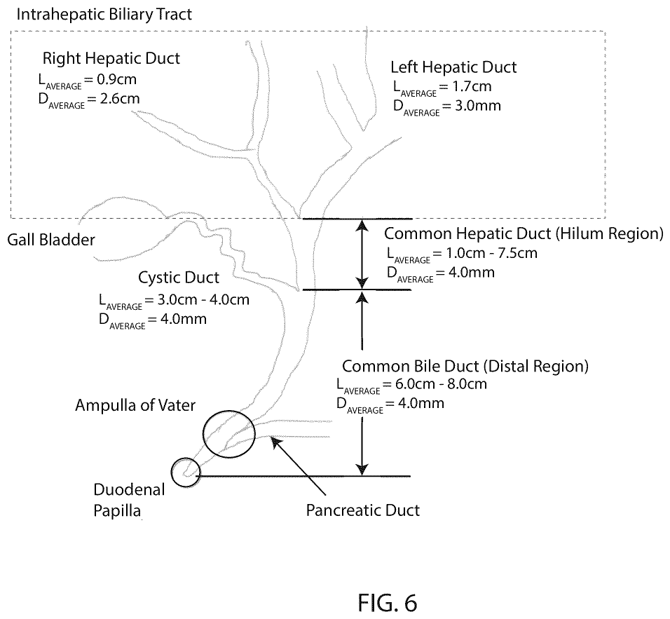

### Treatment Area Examples (FIG. 6)

The cryoablation systems and structures described herein are flexible and well-suited to cryoablate an area along a lumen, vessel, or passageway of the body. The present cryoablation system 100 is configured to be adequately flexible to access and ablate many different such structures. The expansion chamber may be shaped and sized to generate an ice ball or ice cylinder of appropriate geometry for ablating structures along the length of a body lumen. Moreover, the materials and configuration of the probes described herein are selected to protect patient tissue and withstand high operating pressures and low temperatures.

Referring now to FIG. 6, a schematic view of the biliary system is shown in accordance with various embodiments herein. The biliary system includes organs and ducts that make and store bile (a fluid made by the liver that helps digest fat) and release it into the small intestine. The biliary system includes the gallbladder and bile ducts inside and outside the liver.

Bile duct cancer or cholangiocarcinoma is a rare disease in which cancer cells form in the bile ducts. Treatment outcomes for cholangiocarcinoma are generally poor. Current treatment options such as a Whipple procedure or biliary drains are high risk and often ineffective.

Cryoablation is a promising treatment for cholangiocarcinoma. The present cryoablation system 100 is configured to be adequately flexible to access and ablate the bile ducts of a patient. The expansion chamber may be shaped and sized to generate an ice ball of appropriate geometry for ablating tumors in the bile ducts.

In addition to the treatment of cholangiocarcinoma, the cryoablation system 100 can be used to treat a number of conditions including other cancerous tumors (e.g., skin, liver, kidney, bone, lung, prostate and breast), pain, skin conditions (e.g., atypical moles, warts, skin tags or actinic keratosis), arrythmia, or the like. The cryoablation system 100 can additionally ablate benign masses, soft tissue, and healthy tissue.

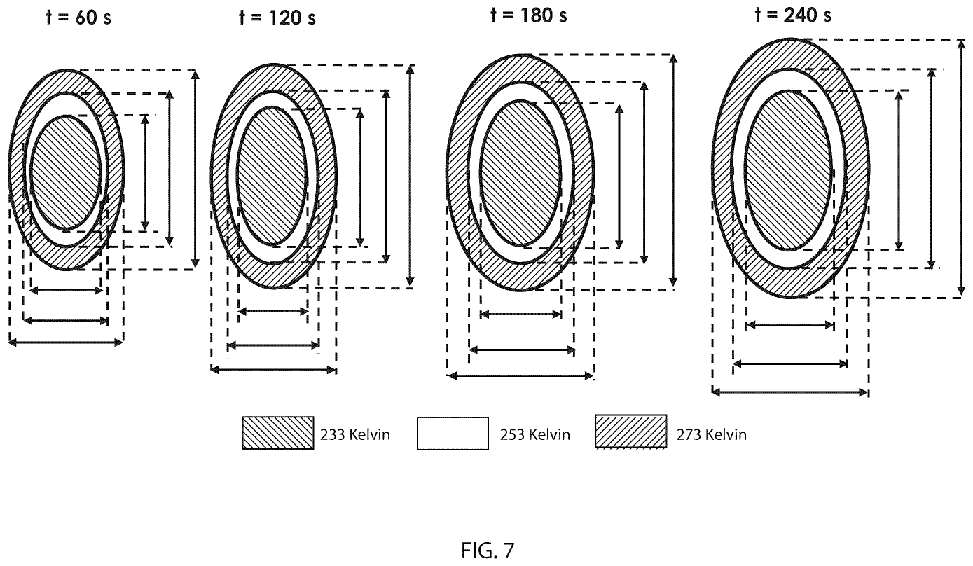

### Ice Ball Formation (FIG. 7)

Referring now to FIG. 7, a schematic view of the growth of an ice ball generated by a cryoablation system is shown in accordance with various embodiments herein. Cryoablation is defined as cell destruction using cold temperatures. An ice ball is formed at the expansion chamber of a cryoablation system, which freezes intra cellular and extracellular material to temperatures colder than 173 Kelvin. The application of these extremely cold temperatures causes cell death. In order to cause complete cell death, sufficiently low ablation temperatures are achieved. Lethal temperatures for various tissues have been reported between 253 Kelvin and 233 Kelvin.

FIG. 7 illustrates the growth of an ice ball over the course of a cryoablation procedure. The time points in this example are at 60 seconds, 120 seconds, 180 seconds, and 240 seconds. Isotherms for 273 Kelvin, 253 Kelvin and 233 Kelvin are depicted for each period of time. As the cryoablation procedure progresses in time, the overall ice ball size increases. More critically, the cell killing isotherms (the 253 Kelvin and 233 Kelvin isotherms) grow along both the major and minor axes of the ice ball.

It should be noted that the times and temperatures given by FIG. 7 are for exemplary purposes only. Ice ball formation can vary based on the configuration of the cryoablation system and the patient's tissue. Moreover, ice ball growth can stabilize after a certain ablation time has been reached. For example, in some embodiments, an ice ball can reach its maximum diameter after ablating the tissue for approximately 10 minutes. In some embodiments, the cryoablation system operator can terminate a cryoablation procedure upon the ice ball reaching its maximum size. In some embodiments, the cryoablation system operator can continue the cryoablation procedure upon the ice ball reaching its maximum size for a predetermined amount of time, as holding the tissue at cryogenic temperatures for longer times may have therapeutic benefits.

In some embodiments, the ice ball is produced and then thawed, and then produced again. In some embodiments, active thawing techniques are used while in other embodiments passive thawing is used.

In the example of FIG. 7, the ice balls are oblong in shape (e.g., they are longer along a primary axis than a secondary axis). The primary axis of the ice ball corresponds to a lengthwise axis of the expansion chamber. The ratio of expansion chamber height to diameter can be correlated to ice ball shape. In particular, an expansion chamber that is much longer than it is wide will result in a more oblong ice ball. Such an ice ball is generally more suitable for the treatment of cholangiocarcinoma, as it is more compatible with the anatomy of the bile ducts. However, other cryoablation applications may require a more spherical ice ball. In such scenarios, the expansion camber can be modified to have a smaller length to diameter ratio.

In some embodiments, the major axis length of an ice ball can be greater than or equal to 0.5 mm, 2 mm, 4 mm, 6 mm, 8 mm, or 10 mm, or can be an amount falling within a range between any of the foregoing. In some embodiments, the minor axis length of an ice ball can be greater than or equal to 0.5 mm, 2 mm, 4 mm, 6 mm, 8 mm, or 10 mm, or can be an amount falling within a range between any of the foregoing.

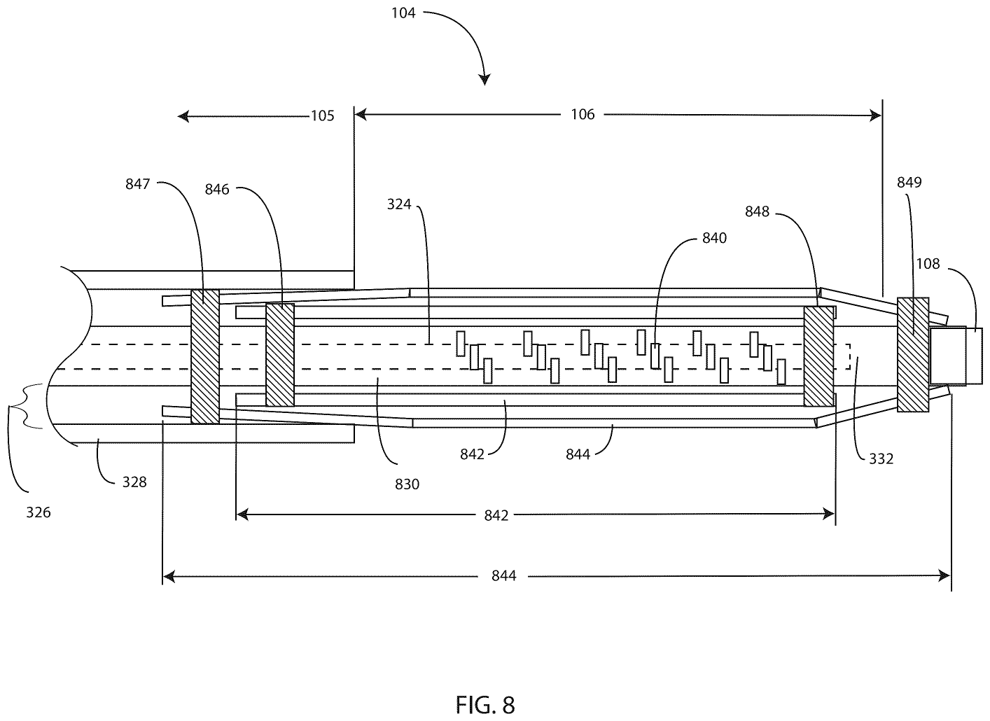

### Cryoablation Shaft Including a Slotted Tube Portion, a Polymer Layer, and a Reinforcing Layer (FIG. 8)

Referring now to FIG. 8, a schematic view of a tip portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 concentrically located within a return tube 326, which is concentrically located within an insulating shaft 328. The concentric-shaft construction is designed to isolate the working gas and active vacuum circuits from each other.

In various embodiments, after exiting the handle 102, the high-pressure flow of the working fluid travels down the supply tube 324. The high-pressure stream of the working fluid may undergo expansion at or downstream of the Joule-Thomson orifice 332 and returns down the shaft in the annular space between the supply tube 324 and the return tube 326.

In various embodiments, the expansion chamber 106 of shaft 104 includes a supply tube 324 concentrically located within the return tube 326. The return tube 326 includes multiple layers in various embodiments.

In various embodiments, the innermost layer of the return tube is a slotted tube 830 which includes slots 840 along at least a portion of the length of shaft 104. Slots 840 are formed in the tube material by any suitable means, such as laser cutting. The slots 840 can be laser cut into the shaft using any suitable pattern to optimize the strength and flexibility of the return tube 326. The material of the slotted tube 830 can be metal such as stainless steel, nitinol, or other durable materials. Many different configurations and patterns of slotted tube 830 are available and one can be selected with the flexibility desirable in this application. The slotted tube forms the core of the return tube 326 in various embodiments.

In some embodiments, the slotted tube 830 includes the slots only in the expansion chamber 106. In this embodiment, the portion of the slotted tube 830 that extends through the insulated zone 105 has solid walls. FIG. 8 illustrates this embodiment by showing a schematic side view of the slotted tube 830, including slots 840 in the expansion chamber 106, and showing the supply tube 324 in dashed lines within the slotted tube 830. In an alternative embodiment, the slotted tube 830 has slots along its entire length.

The slotted tube may be configured to be sufficiently flexible to form a curve having a desired radius of curvature. For instance, the slotted tube may be sufficiently flexible, such that the shaft may form a curve having the smallest radius of curvature of less than or equal to 30 mm, 20 mm, 10 mm, 5 mm, or 3 mm. The slotted tube 830 may have different levels of flexibility in the expansion chamber and the insulated zone 105.

In various embodiments, a first polymer layer 842 surrounds the portion of the slotted tube 830 which includes the slots, in order to contain the working fluid within the shaft 104. In various embodiments, a reinforcing layer 844 surrounds the first polymer layer 842 and is configured to provide additional strength and reinforcement to the first polymer layer 842, thus reducing the likelihood of any leak or rupture in the first polymer layer.

In various embodiments, the first polymer layer 842 can be formed from any suitable polymer such as polyethylene terephthalate (PET), PTFE, ePTFE, PEEK, polyetherimide (PEI), polyimide (PI), or the like. In various embodiments, the reinforcing layer 844 can be a second polymer layer formed from any suitable polymer such as PET, PTFE, PEEK, polyetherimide (PEI), polyimide (PI), or the like. In various embodiments, the reinforcing layer is gas impermeable. In various embodiments, the reinforcing layer 844 is not impermeable and can include a braided polymer material and/or a coiled polymer or metallic material and/or coatings & encapsulants.

In various embodiments, the return tube may include two, three, four, or more polymer layers. In various embodiments, the return tube may include two, three, four, or more total layers.

In the embodiment of FIG. 8, where the slotted portion of the slotted tube 830 is within the expansion chamber, the first polymer layer 842 and the reinforcing layer 844 extend from just within the insulated zone 105 to the distal end. In various embodiments, the first polymer layer 842 is bonded to the slotted tube 830 at first bond 846 near the proximal end of the expansion chamber 106 and at a second bond 848 near the tip 108 of the shaft 104.

In various embodiments, the reinforcing layer 844 is bonded to the slotted tube 830 at first bond 847 near the proximal end of the expansion chamber 106 and/or at a second bond 849 near the tip 108 of the shaft 104.

Additional bonds can be placed along any other suitable location of the shaft. The bonds can be formed from any suitable material or materials such as Vectran, UHMWPE,

PEEK, Polyimide, Metallic Wire. Vectran is a manufactured fiber, spun from a liquid-crystal polymer that displays increased tensile strength at cold temperatures, making it advantageous for use in cryoablation systems. In alternative embodiments, the bonds 846, 847, 848 and 849 may be formed from alternate techniques such as crimp or swage rings, polymer reflow joints, adhesive joints, solder joints or the like.

In various embodiments, the wrap materials of the bonds 846, 847, 848 and 849, can also be wrapped along the entire length of the expansion chamber around the polymer layer, the reinforcing layer, or both. The wrap layer may have a higher pitch of wraps at the bond location and extend along the remainder of the expansion chamber at a lower pitch level. The wrap may extend from the proximal end to the distal end and then reverse direction and extend back in a proximal direction. The number of wrap layers may be one, two, three, four or more.

In various embodiments, the bonds 846, 847, 848 and 849 are configured to increase the burst strength of the layers of the return tube 326.

In some embodiments, the burst strength of each of the first polymer layer 842 and the reinforcing layer 844 can be greater than or equal to 12.4 MPa, 13.1 MPa, 13.8 MPa, 14.5 MPa, 15.2 MPa, or 41.4 MPa. In some embodiments, the burst strength of each of the first polymer layer 842 and the reinforcing layer can be less than or equal to 41.4 MPa, 20.7 MPa, 19.3 MPa, 17.9 MPa, 16.5 MPa, or 15.2 MPa. In some embodiments, the burst strength can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of the bonded first polymer layer 842 and the reinforcing layer 844 together can be greater than or equal to 12.4 MPa, 13.1 MPa, 13.8 MPa, 14.5 MPa, 15.2 MPa 27.6 MPa, or 41.4 MPa. In some embodiments, the burst strength of the bonded first polymer layer 842 and the reinforcing layer 844 together can be less than or equal to 41.4 MPa, 20.7 MPa, 19.3 MPa, 17.9 MPa, 16.5 MPa, or 15.2 MPa. In some embodiments, the burst strength of the bonded first polymer layer 842 and the reinforcing layer 844 together can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of each of the bonds 846, 847, 848 and 849 can be greater than or equal to 1.4 MPa, 4.8 MPa, 8.3 MPa, 11.7 MPa, 15.2 MPa, 27.6 MPa, or 41.4 MPa. In some embodiments, the burst strength of each of the bonds can be less than or equal to 41.4 MPa, 34.8 MPa, 28.3 MPa, 21.7 MPa, or 15.2 MPa. In some embodiments, the burst strength of each of the bonds can fall within a range of 1.4 MPa to 41.4 MPa, or 4.8 MPa to 34.8 MPa, or 8.3 MPa to 28.3 MPa, or 11.7 MPa to 21.7 MPa, or can be about 15.2 MPa.

In some embodiments, the bonds 846, 847, 848 and 849 can have the same burst strength. Alternatively, some of the bonds can have a higher burst strength than others. For instance, proximal bonds 846, 847 may have a lower burst strength than distal bonds 848, 849.

In various embodiments, the return tube 326 may be configured to be sufficiently flexible to form a curve having a desired radius of curvature. For instance, the return tube may be sufficiently flexible, such that the shaft may form a curve having the smallest radius of curvature of less than or equal to 30 mm, 20 mm, 10 mm, 5 mm, or 3 mm.

### Slotted Tube Portion Can Extend the Entire Length of the Return Tube

FIG. 8 illustrates that the slotted tube 830 includes the slots only in the expansion chamber 106, but in an alternative embodiment, the slotted tube 830 has slots along its entire length. In this alternative embodiment, the first polymer layer 842 and the reinforcing layer 844 will also extend along the entire length of the return tube 326. The reinforcing wrap layer, such as a wrap of a Vectran material, may also extend along the entire return tube length. The presence of slots along the entire return tube length may be desirable to achieve the desired level of flexibility along the return tube.

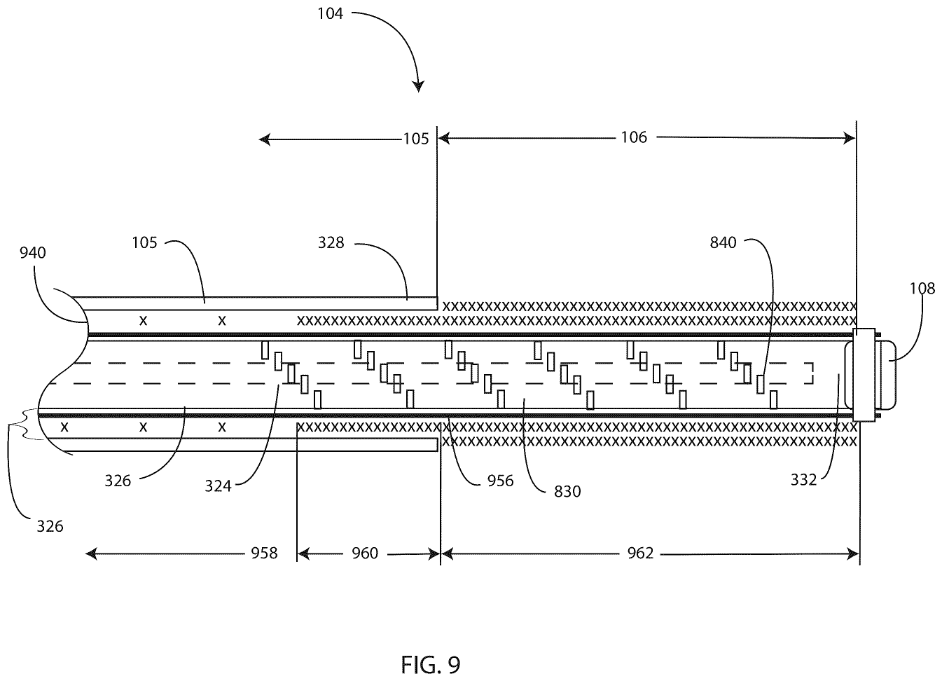

### Cryoablation Shaft Including a Slotted Tube Portion and a Gradient Braid (FIG. 9)

Referring now to FIG. 9, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 located within a return tube 326. The return tube 326 of FIG. 9 includes multiple layers.

The innermost layer of the return tube of 326 is a slotted tube 830, which can be constructed using the options and details described herein. The slotted tube 830 is surrounded by a polymer layer 956, which is concentrically surrounded by a reinforcing gradient braid layer 940.

In various embodiments, gradient braid layer 940 includes zones of different density of braid, increasing in density toward the distal end of the device. A first braid zone 958 is present in the insulated zone and is the least dense. A second braid zone 960 overlaps the insulated zone 105 and the expansion chamber 106 and is more dense than the first braid zone 958. The third braid zone 962 is the densest and is present in the expansion zone. The return tube may include one, two, three, or more braid layers.

In various embodiments, the polymer layer 956 is configured to contain the working fluid in the return tube and not allow the working fluid to escape radially through the slots in the slotted tube. The polymer layer may be constructed from the options and materials discussed herein with respect to the first polymer layer herein. In some embodiments, the expansion chamber 106 may include an additional braided layer between the slotted tube 830 and the polymer layer 956 (not shown in this view). The additional braided layer is configured to prevent friction between the polymer layer 956 and the slotted tube 830.

### Braid Materials, Braid Element Diameters, Braid Density Zones, Coils, and Burst Strength Examples

Braided tubes are used in a variety of medical applications. Braid reinforced tubing can improve functional properties such as strength, stiffness, burst pressure resistance, torque transmission, and kink resistance of a medical device. Such features enable can enable a cryoablation shaft to navigate tortuous portions of a patient's anatomy, such as the biliary ducts. Design considerations such as braid pattern, pick count (ppi), material, wire dimension, wire size/shape and durometer of plastics can have significant impact on device performance.

The braided portions of the shaft can be formed from any suitable material or materials such as metals (e.g., nitinol, stainless steel, tungsten, MP35N, or other such materials), polymers, (e.g., PET, Kevlar, Carbon fiber, Vectran, or other such materials), or the like. Braided materials are formed by weaving a metal or fiber filament in a braid pattern. In various embodiments, the cross section of the filament is circular, however other cross-sectional shapes are possible (e.g., flat, star, triangle). In some embodiments, the diameter of the filament can be greater than or equal to 0.01 mm, 0.1 mm, 0.2 mm, 0.3 mm, or 0.5 mm. In some embodiments, the diameter of the filament can be less than or equal to 2 mm, 1.6 mm, 1.2 mm, 0.8 mm, or 0.5 mm. In some embodiments, the diameter of the filament can fall within a range of 0.01 mm to 2.00 mm, or 0.1 mm to 1.6 mm, or 0.2 mm to 1.2 mm, or 0.3 mm to 0.8 mm, or can be about 0.5 mm.

In various embodiments, the density of the braided portions can be altered with more dense braids offering more radial strength and rigidity and less dense braids offering more flexibility. The shaft can include multiple braids having varying material properties. In various embodiments, the shaft may include a light braid (e.g., have relatively low braid density) between the return tube 326 and the polymer layer 956. As described above, the light braid may offer limited structural support but can prevent undue friction between the slotted return tube 326 and the polymer layer 956.

In various embodiments, the density of the braided materials can increase from the base of the shaft 104 to the tip 108 of the shaft. In the event that there is a device failure, such a configuration improves the likelihood of the shaft failing closer to the base. Such a failure mode is generally preferable compared to a failure closer to the tip 108 of the shaft in terms of patient safety outcomes.

In various embodiments, the first gradient braid zone 958 spans the insulated zone 105 of the shaft 104 (starting from where the shaft connects to the handle 102 and terminating at or before the expansion chamber 106). In various embodiments, the gradient braid 958 portion can have a first burst strength. The first burst strength can be constant along the insulated zone of the shaft. Alternatively, the first burst strength of the gradient braid portion 958 can increase from the base of the shaft to the expansion chamber. In various embodiments, the gradient braid portion 958 can have a first braid density. The first braid density can be constant along the insulated zone of the shaft. Alternatively, the first braid density of the gradient braid portion 958 can increase from the base of the shaft to the expansion chamber.

In some embodiments, the minimum burst strength of the gradient braid portion 958 can be less than or equal to 20.7 MPa, 13.8 MPa, 6.9 MPa, 5.5 MPa, 4.1 MPa, 2.8 MPa, 1.4 MPa, or 0.7 MPa, or can be an amount falling within a range between any of the foregoing. In some embodiments, the maximum burst strength of the gradient braid portion 958 can be greater than or equal to 0.7 MPa, 2.0 MPa, 3.4 MPa, 4.8 MPa, 6.2 MPa, 6.9 MPa, 13.8 MPa, or 20.7 MPa or can be an amount falling within a range between any of the foregoing.

In various embodiments, the second braid portion 960 spans the expansion chamber 106 of the shaft 104. In some embodiments, the second braid portion 960 may start at the beginning of the expansion chamber and terminate at or near the tip 108 of the shaft. Alternatively, the second braid portion 960 may start towards the distal end of the insulated zone 105 of the shaft (as depicted by FIG. 9) and terminate at or near the tip 108 of the shaft. In some embodiments, the second braid portion 960 is a continuation of the first braid portion 958. Alternatively, the second braid portion 960 may be made of elements that are physically separate from the elements of first braid zone 958.

In various embodiments, the second braid portion 960 can have a second burst strength. The second burst strength can be constant along the length of the first braid layer. Alternatively, the second burst strength of the second braid portion 960 can increase from the along the length of the first braid layer (from the end of the insulated zone 105 to the tip 108). In various embodiments, the second braid portion 960 can have a second braid density. The second braid density can be constant along the length of the second braid portion. Alternatively, the second braid density of the second braid portion 960 can increase along the length of the second braid portion.

In some embodiments, the burst strength of the second braid portion 960 can be greater than or equal to 10.3 MPa, 12.9 MPa, 15.5 MPa, 18.1 MPa, or 20.7 MPa, or 41.4 MPa. In some embodiments, the burst strength of the second braid portion 960 can be less than or equal to 34.5 MPa, 31.0 MPa, 27.6 MPa, 24.1 MPa, or 20.7 MPa. In some embodiments, the burst strength of the second braid portion 960 can fall within a range of 10.3 MPa to 34.5 MPa, or 12.4 MPa to 31.0 MPa, or 15.5 MPa to 27.6 MPa, or 17.9 MPa to 34.5 MPa, or can be about 20.7 MPa.

In various embodiments, the third braid zone 962 spans the expansion chamber 106 of the shaft 104. In some embodiments, the third braid zone 962 may start at the beginning of the expansion chamber and terminate at or near the tip 108 of the shaft.

In various embodiments, the third braid zone 962 can have a third burst strength. The third burst strength can be constant along the length of the third braid zone. Alternatively, the burst strength of the third braid zone 962 can increase from the along the length of the third braid zone. In various embodiments, the third braid zone 962 can have a third braid density. The braid density can be constant along the length of the third braid zone. Alternatively, the braid density of the third braid zone 962 can increase along the length of the first braid layer.

In some embodiments, the burst strength of the third braid zone 962 can be greater than or equal to 10.3 MPa, 12.9 MPa, 15.5 MPa, 18.1 MPa, or 20.7 MPa. In some embodiments, the burst strength of the third braid zone 962 can be less than or equal to 34.5 MPa, 31.0 MPa, 27.6 MPa, 24.1 MPa, or 20.7 MPa. In some embodiments, the burst strength of the third braid zone 962 can fall within a range of 10.3 MPa to 34.5 MPa, or 12.4 MPa to 31.0 MPa, or 15.5 MPa to 27.6 MPa, or 17.9 MPa to 24.1 MPa, or can be about 20.7 MPa.

In alternative embodiments, any, or all of the gradient braid zones 958, 960, 962 can be formed from coils. A coil, as defined herein is a filament of material wound around the shaft. Like in braided materials, as detailed above, the filament can be selected to have suitable material(s) and cross-sectional shapes including round, rectangular, or other shapes. The zones can include coils of different density, radial strength, rigidity, and flexibility. The coils can be single layered or multi layered. In some embodiments, the multi-layer coils can have alternate wind directions (e.g., first layer is wound clockwise, and the second layer is wound counterclockwise). In various embodiments, the pitch of the coil can be varied to optimize the properties of the shaft, such as flexibility, burst strength, or the like. For instance, tighter pitches of coil can increase the burst strength of the shaft while looser pitches of coil can increase the flexibility of the shaft. In various embodiments, the pitch of the coil materials can increase from the base of the shaft 104 to the tip 108 of the shaft.

### Transitions Between Braid Zones

In some embodiments, the transitions between the first, second and third braid portions are transitions in density of the same physical braid elements, so that one braid zone is a continuation of a neighboring braid zone. Alternatively, one braid portion may be made of elements that are physically separate from the elements of a neighboring braid zone.

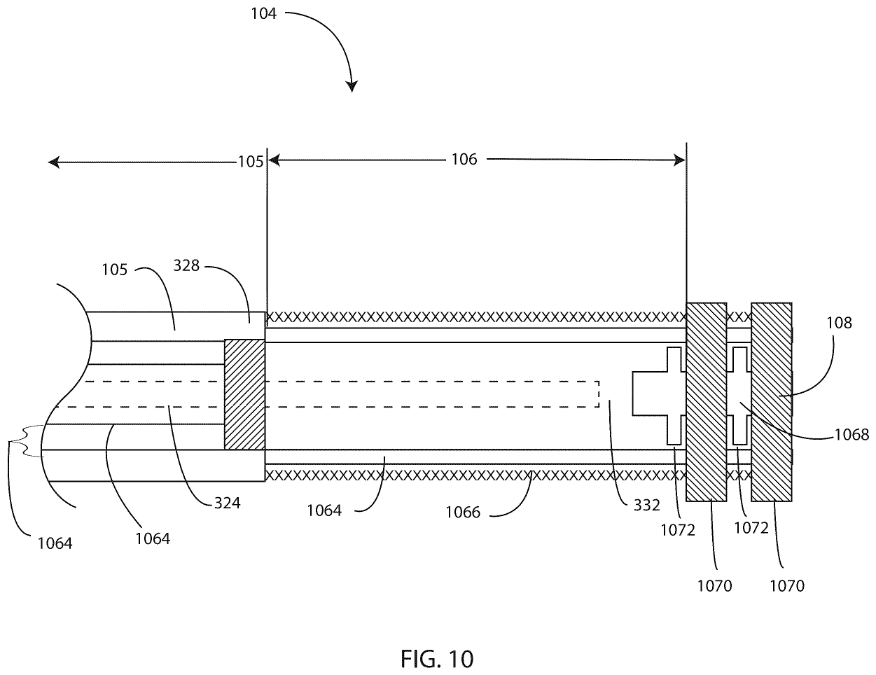

### Composite Shaft (FIG. 10)

Referring now to FIG. 10, a schematic view of a portion of a cryoablation shaft is shown in accordance with various embodiments herein. In various embodiments, the expansion chamber 106 of shaft 104 includes a supply tube 324, located concentrically within a composite return tube 1064 In various embodiments, the return tube can be a composite shaft. In various embodiments, the composite return tube 1064 is formed from one or more discrete layers. One possible return tube layer is a braid layer 1066 shown in FIG. 10.

In various embodiments, composite return tube 1064 can be formed from one or more polymer and/or braided layers as described in detail herein. After forming the discrete layers, the composite shaft may be heated to such a temperature that the discrete layers bond to each other forming a single composite layer. In some embodiments, some of the discrete layers may contribute to the radial strength of the expansion chamber and other discrete layers may contribute to the gas containment capability of the expansion chamber. In such embodiments, radial strength and gas containment may be achieved from a single composite layer. Such a configuration can enhance the tunability of the expansion chamber material properties while decreasing the diameter of the expansion chamber.

In some embodiments, an additional braid layer 1066 may be disposed over the remainder of the composite return tube 1064. The additional braid layer is selected to have braid properties that enhance the radial strength of the expansion chamber 106.

### Distal Fitting Examples (FIG. 10)

In various embodiments, the working fluid may be contained at the tip 108 of the shaft using a plug 1068 and one or more joints 1070. In various embodiments, the joints 1070 and/or the plug 1068 may be of a material or materials that are compatible to bond with the composite return tube 1064 and/or the braid layer 1066 to enhance the seal of the shaft 104. In various embodiments, the joints 1070 and/or the plug 1068 may be of a material or materials that are sufficiently strong as to enhance the burst strength of the expansion chamber.

In various embodiments, plug 1068 includes ridges 1072. The ridges 1072 improve the mechanical strength of the bonded joints 1070 by providing additional structural support to the bonds to resist the pressure within the expansion chamber.

### Markers for Imaging Systems

The joint 334 (FIG. 3) and the distal tip 108 (FIGS. 8 and 10) are configured to appear on imaging systems. The joint 334 can include a marker band of radiopaque materials, such as tungsten or platinum iridium or other appropriate materials. The material and dimensions of the distal tip 108 can be configured to appear on imaging systems. Examples of materials for the distal tip 108 include stainless steel and nitinol. The markers at either end of the active zone of the device assist the physician with visually identifying the location of the active zone of cryoablation with respect to the patient's anatomy and the target zone for cryoablation. Examples of imaging systems include ultrasound, fluoroscopy, cone beam CT, and MRI systems.

### Introduction Options

In some embodiments, the catheter system is delivered using a sheath introduction system. An example of a sheath introduction system is a steerable sheath. Alternatively, the catheter system can be steerable. In another embodiment, the catheter system includes a monorail lumen along a portion of the catheter to facilitate introduction.

### Guidewire Delivery Options

In various embodiments, it is desirable for the cryoablation system to include a guidewire. A guidewire as defined herein is a flexible, medical wire inserted into the body to guide a larger instrument, such as a catheter, to a target location. Implementing a guidewire with a cryoablation system can enable the flexible shaft to access portions of the patient's anatomy (e.g., the biliary system) with increased accuracy and decreased damage. A guidewire may run through a guidewire lumen that extends either part way or all the way though the length of the shaft. A guidewire lumen, as defined herein, is a channel or hollow space that facilitates the insertion of a guidewire. A guidewire lumen can be added to the cryoablation system in either a monorail or an over-the-wire configuration. In alternate embodiments, the cryoablation system may be introduced to a patient using a sheath rather than a guidewire.

In a monorail configuration, the guidewire and guidewire lumen only run through a portion of the length of the shaft. For the case of the cryoablation system 100, the guidewire and guidewire lumen may run only though a distal portion of the shaft 104 (e.g., only through a portion adjacent to or slightly space away from the tip 108) in a monorail configuration. In some embodiments, the distal tip 108 can include a lumen for the guidewire to pass through. Monorail configurations do not require running a guidewire lumen all the way through the shaft resulting in smaller possible shaft diameters and reduced shaft complexity compared to over-the-wire configurations, in which the guidewire lumen runs the entire length of the shaft. In various embodiments, when using a monorail guidewire, most of the cross section of shaft 104 is not altered from the shaft embodiment depicted by FIG. 3.

In an over-the-wire configuration, an additional guidewire lumen is present and extends along the length of the shaft. In various embodiments, the lumen extends along the entire length of the shaft. Over-the-wire catheter configurations are particularly useful in positioning medical devices in difficult to navigate portions of the anatomy (e.g., the biliary system). Compared to monorail lumen configurations, over-the-wire catheter configurations decrease the possibility of uneven strain being placed on portions of the catheter construction during the bending that occurs during the shaft navigation and/or positioning processes, thereby decreasing the chances of structural damage or failure in the catheter device. However, the requirement of running an additional lumen and guidewire through the shaft can increase both the size of the outer diameter and complexity of the cryoablation shaft.

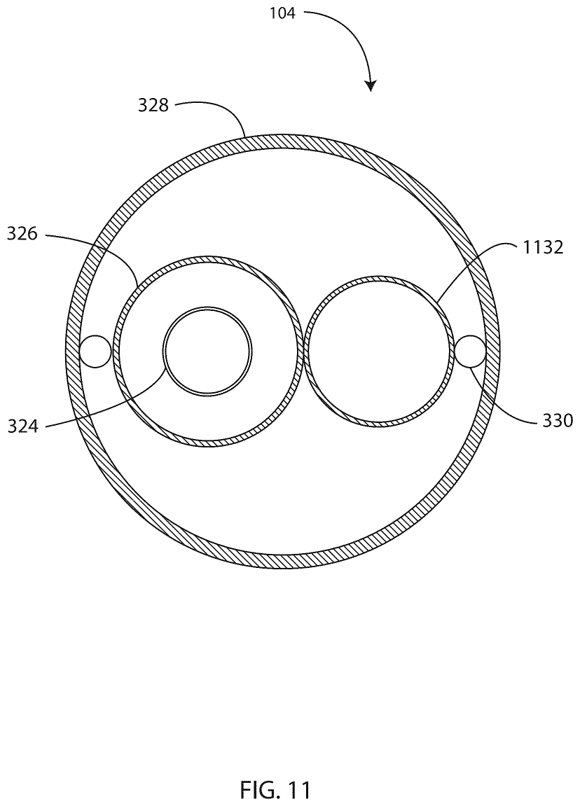

In a particular example, to illustrate the size difference between a monorail shaft and an over-the-wire shaft, the outer diameter of a monorail shaft can be about 6-7 French (2.00 to 2.33 mm) and the outer diameter of an over-the-wire shaft can be about 8-9 French (2.67 to 3.00 mm). These ranges are for exemplary purposes only, but it should be noted that over-the-wire shafts generally require an equal or larger diameter than their monorail counterparts. To implement an over-the wire configuration, shaft 104 of the cryoablation system needs to be altered to accommodate an additional guidewire lumen and guidewire. This may be done in any suitable manner, but three possible examples are illustrated in FIGS. 11-13.

In each of the examples described below with respect to FIGS. 11-13, the outer profile of each of the following elements of the cryoablation system can be circular: supply tube, return tube, guidewire lumen, and insulation shaft. In other words, these elements have a circular outer profile in a radial cross-sectional view. In alternate embodiments, the outer profile of the supply tube, return tube, guidewire lumen, and/or insulation shaft can be any other suitable shape, such as oblong, rectangular, or any other shape that facilitates positioning within a body lumen.

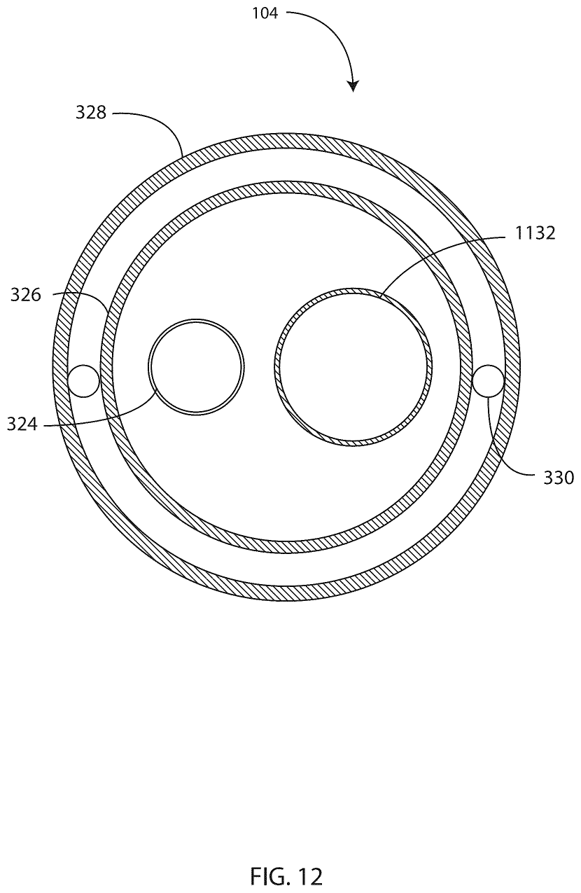

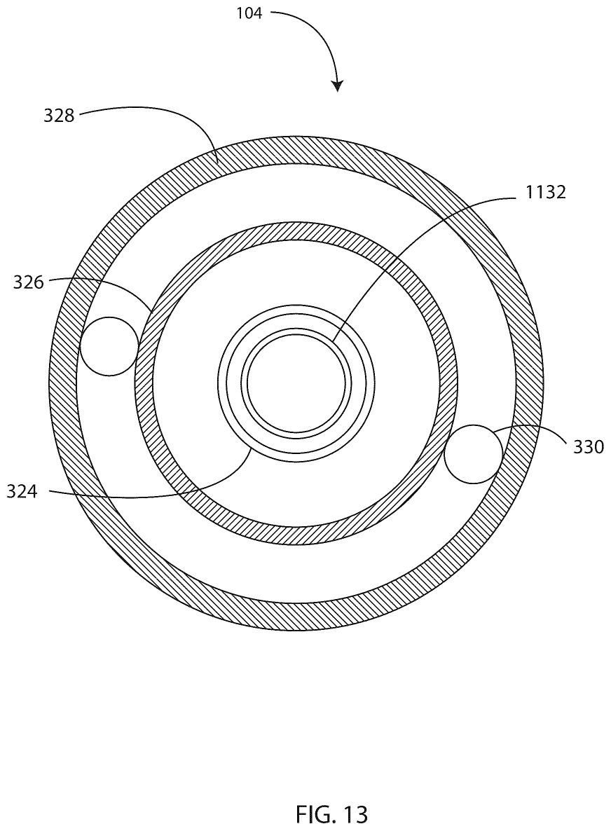

In each of the examples described below with respect to FIGS. 11-13, the inner space defined by the guidewire lumen is circular and does not house any other components other than the guidewire during the introduction process. The guidewire can be removed from the guidewire lumen after introduction of the catheter to a target body location. Alternatively, depending on the procedure to be performed, the guidewire may be left inside the guidewire lumen during the cryoablation procedure. In each of the examples described below with respect to FIGS. 11-13, the supply tube is located within the return tube, either concentrically (FIGS. 11 and 13) or off-center (FIG. 12).

In each of the examples described below with respect to FIGS. 11-13, the inner space of the insulation shaft is occupied by the other elements and, as a result, its inner space used by the vacuum insulation chamber is not circular and varies in its shape between the examples. The inner space defined by the return tube and supply tube also varies between the different examples because the inner spaces defined by these elements are sometimes occupied by one or more of the other elements.

### Isolated Exhaust Configuration (FIG. 11)

Referring now to FIG. 11, a cross sectional view of a cryoablation shaft is shown in accordance with various embodiments herein. In various embodiments, the shaft 104 may include a supply tube 324, return tube 326, insulation shaft 328, and filament 330. The shaft also includes a guidewire lumen 1132 extending throughout the length of the shaft 104 for the insertion of a guidewire. In the example of FIG. 11, the supply tube 324 is concentrically located within the return tube 326 and the guidewire lumen 1132 is tangent to the return tube 326. The guidewire lumen 1132 and return tube 326 are each tangentially connected to the insulation shaft 328 by a supporting filament 330. The supply tube 324, return tube 326, guidewire lumen 1132, and filament 330 are all enclosed by the insulation shaft 328. In alternate embodiments, the shaft may not include filament 330.

In various embodiments, after exiting the handle 102, the high-pressure flow of the working fluid travels distally down the shaft 104 through supply tube 324. After cooling and expansion in the expansion chamber 106, the working fluid travels proximally back through the shaft 104 in the annular space between the supply tube 324 and return tube 326.

In the example of FIG. 11, the supply tube has a substantially circular cross section and does not house any other elements. In the example of FIG. 11, the supply tube 324 is housed concentrically within the return tube 326.

In the example of FIG. 11, the return tube 326 and guidewire lumen 1132 are positioned to be adjacent, side-by-side to each other. The outer profile of the return tube 326 combined with the guidewire lumen 1132 is similar to the outer profile of a Figure-8 shape.

The vacuum chamber space is defined between the inner wall of the insulation shaft 328 and the outer walls of the return tube 326 and guidewire lumen 1132. The vacuum chamber space is a circle without the inner Figure-8 space occupied by the guidewire lumen 1132 and return tube 326.

The supporting filament 330 is wound around the outer surfaces of the supply tube 324 and the guidewire lumen 1132, holding those two components in a fixed relationship to each other. In alternate embodiments, the shaft may not include filament 330.

### Common Exhaust Configuration (FIG. 12)

Referring now to FIG. 12, a cross sectional view of a cryoablation shaft is shown in accordance with various embodiments herein. In various embodiments, the shaft may include a supply tube 324, return tube 326, insulation shaft 328, and a supporting filament 330. The shaft also includes a guidewire lumen 1132 extending throughout the length of the shaft 104 for the insertion of a guidewire. In the example of FIG. 12, the return tube 326 is concentrically located within the insulation shaft 328 and the filament 330 is disposed between return tube 326 and the insulation shaft 328. The supply tube 324 and the guidewire lumen 1132 are both disposed within the return tube 326. In alternate embodiments, the shaft may not include filament 330.

In various embodiments, after exiting the handle, the high-pressure flow of the working fluid travels distally down the shaft 104 through supply tube. After cooling and expansion in the expansion chamber 106, the working fluid travels proximally back through the shaft 104 in the space between the supply tube 324 and return tube 326. As can be seen in the example of FIG. 12, the guidewire lumen 1132 is also positioned in the space between the supply tube 324 and return tube 326. As such, the return passage for the working fluid is defined by the cross-sectional area of the return tube 326 minus the cross-sectional area of the guidewire lumen 1132 and the cross-sectional area of the supply tube 324.

In the example of FIG. 12, the supply tube 324 and guidewire lumen 1132 both have a circular cross-section and do not house any other components. The supply tube 324 and guidewire lumen 1132 are located side-by-side. In some embodiments, the supply tube 324 and guidewire lumen 1132 are adjacent to each other.

In the example of FIG. 12, the supply tube 324 and guidewire lumen 1132 are both positioned within the return tube 326. The return tube 326 defines the space for working gas to return, and that space has a circular outer profile, with an inner profile defined around the circular profiles of the supply tube 324 and guidewire lumen 1132.

The supply tube 324 and guidewire lumen 1132 are positioned to be side-by-side with each other. There may or may not be a gap between the supply tube 324 and the guidewire lumen 1132. In various embodiments, the supply tube 324 and guidewire lumen 1132 are adjacent to each other, and the outer profile of the supply tube 324 combined with the guidewire lumen 1132 is similar to the outer profile of a Figure-8 shape.

The working gas return space within the return tube 326 is defined between the inner wall of the return tube 326 and the outer walls of the supply tube 324 and guidewire lumen 1132. The working gas return space is defined around the inner space, which may be occupied by the guidewire lumen 1132 and supply tube 324, with a circular outer profile.

The return tube 326 is located concentrically within the insulation shaft 328. The vacuum chamber space is annular, between the insulation shaft inner wall and the return tube outer wall.

### Central Guidewire Lumen Configuration (FIG. 13)

Referring now to FIG. 13, a cross sectional view of a cryoablation shaft is shown in accordance with various embodiments herein. In various embodiments, the shaft 104 may include a guidewire lumen 1132 concentrically located within a supply tube 324, which is concentrically located within a return tube 326, which is concentrically located within an insulation shaft 328, with the filament 330 disposed between the return tube and the insulation shaft. In alternate embodiments, the shaft may not include filament 330.

In various embodiments, after exiting the handle 102, the high-pressure flow of the working fluid travels distally down the shaft 104 through supply tube 324. As the guidewire lumen 1132 is located concentrically within the supply tube 324, the working gas fluid supply travels down the annular space between the return tube 326 and the guidewire lumen 1132. After cooling and expansion in the expansion chamber 106, the working fluid travels proximally back through the shaft 104 in the annular space between the supply tube 324 and return tube 326.

In the example of FIG. 13, the supply tube 324 is located concentrically within the return tube 326. The return tube 326 is located concentrically within the insulation shaft 328. The vacuum chamber space is annular, between the insulation shaft inner wall and the return tube outer wall.

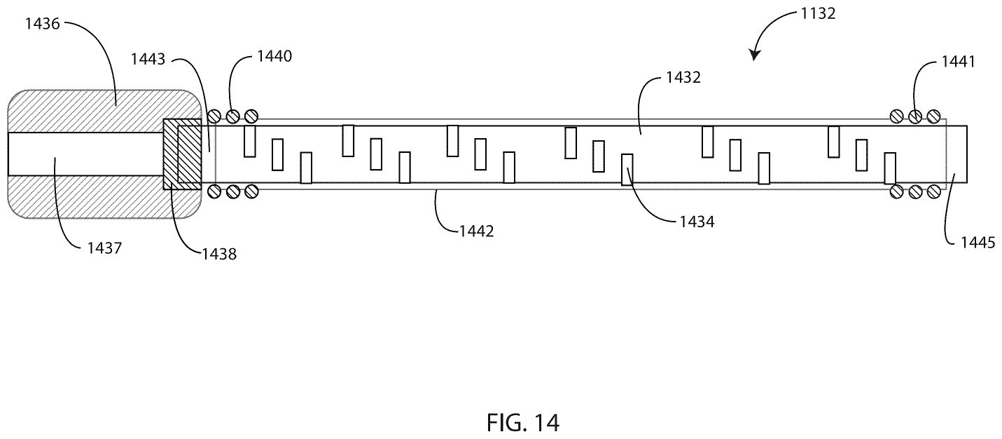

### Guidewire Lumen Construction (FIG. 14)

Referring now to FIG. 14, a cross sectional view of a guidewire lumen is shown in accordance with various embodiments herein. In various embodiments, when inserted into a cryoablation shaft, the guidewire lumen 1132 is configured to extend through the length of the cryoablation shaft 104. In various embodiments, the guidewire lumen 1132 is configured for insertion of a guidewire. In various embodiments, the guidewire lumen 1132 can include a metallic tube 1432 and a polymer sleeve 1442 configured to surround at least a portion of the metallic tube.

In various embodiments, the guidewire lumen 1132 can include a metallic tube 1432. The metallic tube can be formed from any suitable material or material such as nitinol, stainless steel, or the like. In various embodiments, at least a portion of the metallic tube 1432 can include one or more slots 1434. Slots 1434 can be formed in the tube material by any suitable means, such as laser cutting or the like. The slots 1434 can be laser cut into the metallic tube 1432 using any suitable pattern to optimize the strength and flexibility of the metallic tube 1432. Many different configurations and patterns of slots 1434 are available and one can be selected with the flexibility desirable in this application.

In some embodiments, the metallic tube 1432 can contain slots 1434 along its entire length. In some embodiments, the metallic tube 1432 can define slots 1434 along its length between portions of metallic tube 1432 that do not contain slots. For instance, the metallic tube 1432 may be devoid of slots 1434 at a distal end portion 1443 at its distal end. In various embodiments, the metallic tube 1432 may be devoid of slots 1434 at a proximal end portion 1445 located at its proximal end. In various embodiments, the metallic tube 1432 may be devoid of slots 1434 at both a distal end portion 1443 and at a proximal end portion 1445.

The metallic tube 1432 may be configured to be sufficiently flexible to form a curve having a desired radius of curvature. For instance, the metallic tube 1432 may be sufficiently flexible, such that the guidewire lumen 1132 may form a curve having the smallest radius of curvature of less than or equal to 30 mm, 20 mm, 10 mm, 5 mm, or 3 mm. The flexibility of the metallic tube 1432 enables the overall cryoablation shaft to have a flexibility that allows steering to treatment sites.

In various embodiments, the guidewire lumen 1132 can include a polymer sleeve 1442. The polymer sleeve 1442 can be formed form any suitable material or material such as polyether block amide, polyethylene terephthalate, or the like. In various embodiments, the polymer sleeve 1442 is configured to cover at least a portion of the metallic tube 1432. In the example of FIG. 14, at least a portion of the metallic tube 1432 includes slots 1434 and the polymer sleeve 1442 is configured to form a seal around the slotted portion of the metallic tube 1432. In such an embodiment, the polymer sleeve 1442 is configured to provide a gas-tight seal around the metallic tube 1432, preventing gases from entering the metallic tube through the slots while maintaining the flexibility of the metallic tube.

In various embodiments, the polymer sleeve 1442 covers the entire length of the metallic tube 1432. Alternatively, such as shown in FIG. 14, the polymer sleeve 1442 does not cover the metallic tube 1432 at a distal end portion 1443. In various embodiments, the polymer sleeve 1442 does not cover the metallic tube 1432 at a proximal end portion 1445. In various embodiments, the polymer sleeve 1442 does not cover the metallic tube 1432 at both a distal end portion 1443 and at a proximal end portion 1445. In various embodiments, the polymer sleeve 1442 extends along the length of the metallic tube 1432 between the distal end portion 1443 and the proximal end portion 1445. Exposure of the metallic tube 1432 at either the distal end, the proximal end, or both ends, provides a metallic surface to bond other components to create a gas-tight seal.

In various embodiments, the guidewire lumen 1132 can include a first compressive wrap 1440 configured to seal the polymer sleeve 1442 to the metallic tube 1432 at a distal end of the polymer sleeve 1442. In various embodiments, the guidewire lumen 1132 can include a second compressive wrap 1441 configured to seal the polymer sleeve 1442 to the metallic tube 1432 at a proximal end of the polymer sleeve 1442. In the example of FIG. 14, the polymer sleeve 1442 is sealed to the metallic tube 1432 such that distal end portion 1443 and proximal end portion 1445 of the metallic tube 1432 are not covered by the polymer sleeve or the compressive wrap.

In various embodiments, the first compressive wrap 1440 and the second compressive wrap 1441 are configured to apply a consistent compressive force around the polymer sleeve 1442, sealing the polymer sleeve to the metallic tube 1432.

In various embodiments, the first compressive wrap 1440 and the second compressive wrap 1441 include a filament wrapped around the polymer sleeve 1442 and the metallic tube 1432 of the guidewire lumen 1132. In various embodiments, the filament is configured to be wrapped around the polymer sleeve 1442 and the metallic tube 1432 of the guidewire lumen 1132 with a wrap threshold tension. For instance, the filament should be able to withstand hanging an object of predetermined mass without breaking. The predetermined mass can be 0.5 kg, 1 kg, 1.5 kg, or greater. In some embodiments, the filament is hung with the predetermined mass during the wrapping process to apply sufficient wrap tension.

In various embodiments, the compressive wrap can have a tensile strength. In some embodiments, the tensile strength can be greater than or equal to 1.0 gigapascal (GPa), 1.8 GPa, 2.7 GPa, or 3.5 GPa. In some embodiments, the tensile strength can be less than or equal to 6.0 GPa, 5.2 GPa, 4.3 GPa, or 3.5 GPa. In some embodiments, the tensile strength can fall within a range of 1.0 GPa to 6.0 GPa, or 1.8 GPa to 5.2 GPa, or 2.7 GPa to 4.3 GPa, or can be about 3.5 GPa.

In various embodiments, the compressive wrap can be a monofilament, a multi-filament material, a unifilar material, a multi-filar material, a metal, a plastic, or a composite material. In various embodiments, the compressive wrap is configured to allow termination of the wrap while maintaining tension. In various embodiments, the compressive wraps 1440, 1441 can be formed from monofilament sutures and/or multi-strand sutures. In various embodiments, the compressive wrap can be formed from material or materials including, but not limited to fibrous materials, such as stainless steel, tungsten, platinum-iridium alloys (Pt-Ir), fiber base materials (e.g., Technora, Dektran), or the like.

In various embodiments, the compressive wrap can have a closed pitch (each wind of the filament is touching an adjacent wind). Alternatively, the compressive wrap may have pitch that is greater than or equal to 0.5 mm, 1.0 mm, 1.5 mm, or 2.0 mm, or can be an amount falling within a range between any of the foregoing.

In some embodiments where the compressive wrap is a filament, the diameter of the filament can vary depending on the material of the filament and its ability to hold tension during manufacturing. In some embodiments, the diameter of the filament can be greater than or equal to 0.002 mm, 0.004 mm, or 0.005 mm. In some embodiments, the diameter of the filament can be less than or equal to 0.007 mm, 0.006 mm, or 0.005 mm. In some embodiments, the diameter of the filament can fall within a range of 0.002 mm to 0.007 mm, or 0.004 mm to 0.006 mm, or can be about 0.005 mm.

In various embodiments, the first compressive wrap 1440 and the second compressive wrap 1441 can include an adhesive configured to encapsulate the compressive wrap. The adhesive is configured to maintain the wrap threshold tension and can enhance the material properties of the compressive wrap such as biocompatibility, radiopacity, or the like. Alternatively, the adhesive can be applied to discrete portions of compressive wraps 1440, 1441, such as at the ends. Examples of suitable adhesive or adhesives that can be used to encapsulate the compressive wrap include cyanoacrylate adhesive and LOCTITE® adhesive available from Henkel Adhesive Technologies, which has a location in Bridgewater, New Jersey, USA, or the like.

In various embodiments, the filament of the first compressive wrap 1440 can be formed from a material or materials configured to transmit electrical signals (e.g., conductive wire). In such an embodiment, one or more sensors can be placed at the distal end of the shaft 104 and the sensor data can be conveyed from the sensor(s) to the console via the electrically conductive filament. In one example, an electromagnetic position sensor can be included at the distal tip 1436 of the shaft 104 and is configured to aid the physician in positioning the shaft within the patient's anatomy. In such a configuration, the first compressive wrap 1440 can be configured to carry a low voltage that would be translated into a position from the distal tip 1436 to the console. In another example, the first compressive wrap 1440 could incorporate fiber optic cable(s) configured to provide information regarding the shape of the target anatomy. Any other suitable sensor or combination of sensors can be included at the distal end of the shaft including but not limited to temperature sensors, pressure sensors, strain sensors, or the like.

In various embodiments, the filament of the first compressive wrap 1440 can be formed from one or more high resistance wires. The high resistance wires can be configured to enable electrical heating of the expansion chamber 106 of the shaft 104. The electrical heating can facilitate more rapid thawing of ice balls, which can decrease cryoablation procedure time and reduce the likelihood of tract seeding, where harmful tissue adheres to the shaft be distributed to other portions of a patient's anatomy.

In various embodiments, the guidewire lumen 1132 is configured to seal to a distal tip 1436 of a cryoablation shaft 104. In various embodiments, the distal tip 1436 is configured to seal a distal end of the expansion chamber 106 of the cryoablation shaft 104. The distal tip 1436 can be formed form any suitable material or material such as nitinol, stainless steel, or the like.

In various embodiments, distal end portion 1443 of the metallic tube 1432 is joined to a proximal end of the distal tip 1436 using a metal-to-metal joining process such as welding, soldering, brazing, or the like. In the example of FIG. 14, the distal end portion 1443 of the metallic tube is devoid of slots 1434 and is not covered by the polymer sleeve 1442. Such an embodiment allows for the distal end portion 1443 of the metallic tube 1432 to form a robust metal-to-metal joint 1438 with the distal tip 1436.

In various embodiments, the distal tip 1436 defines a central channel 1437. The central channel 1437 is configured to receive a guidewire. In various embodiments, a proximal end of the central channel 1437 of the distal tip 1436 can be joined to the distal end portion 1443 of the metallic tube 1432 to form the metal-to-metal joint 1438.

In various embodiments, the proximal end portion 1445 of the metallic tube 1432 can be joined to the cryoablation probe at any suitable location such as at a proximal end of the shaft 104 or at the handle 102.

In various embodiments, the guidewire lumen 1132 is configured to be sufficiently robust as to not be compressed by the high-pressure gas flows in the cryoablation shaft 104. In various embodiments, the guidewire lumen 1132 is configured to withstand compressive pressures during normal operation of the cryoablation system of about 1 MPa or 150 psi. In various embodiments, the burst strength of the guidewire lumen 1132 can be greater than or equal to 0.4 MPa, 0.6 MPa, 0.7 MPa, 0.8 MPa, or 1.0 MPa.

In various embodiments, the guidewire lumen 1132 is configured to be sufficiently robust to withstand larger compressive pressures of about 15 MPa or 2200 psi that might occur during a fault condition of the cryoablation system. In some embodiments, the burst strength of the guidewire lumen 1132 can be greater than or equal to 9.0 MPa, 10.5 MPa, 12.0 MPa, 13.5 MPa, or 15.0 MPa. In some embodiments, the burst strength can be less than or equal to 21.0 MPa, 19.5 MPa, 18.0 MPa, 16.5 MPa, or 15.0 MPa. In some embodiments, the burst strength can fall within a range of 9.0 MPa to 21.0 MPa, or 10.5 MPa to 19.5 MPa, or 12.0 MPa to 18.0 MPa, or 13.5 MPa to 16.5 MPa, or can be about 15.0 MPa.

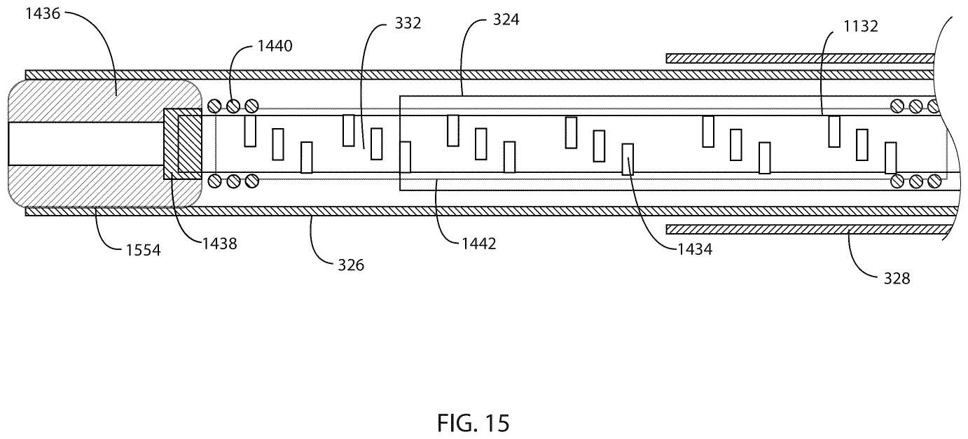

### Central Guidewire Lumen Embodiment with Distal Tip (FIG. 15)

Referring now to FIG. 15, a schematic view of a portion of the guidewire lumen and distal tip of FIG. 14 is shown within a cryoablation shaft in accordance with various embodiments herein. In the example of FIG. 15, the shaft has a central guidewire lumen configuration such as shown and described by FIG. 13. Alternatively, it is possible for the guidewire construction options described with respect to FIG. 14 to be used in an isolated exhaust embodiment of FIG. 11 or in a common exhaust embodiment of FIG. 12.

Referring again to FIG. 15, in various embodiments, the shaft 104 may include a guidewire lumen 1132 concentrically located within a supply tube 324, which is concentrically located within a return tube 326, and at least a portion of the return tube is concentrically located within an insulation shaft 328.

In various embodiments, after exiting the handle 102, the high-pressure flow of the working fluid travels down the supply tube 324. The high-pressure stream of the working fluid may undergo expansion at or downstream of the Joule-Thomson orifice 332 and returns down the shaft in the annular space between the supply tube 324 and the return tube 326.

In various embodiments, after being bonded to the distal tip 1436, the guidewire lumen 1132 is configured to be inserted into the distal end of the shaft 104. In various embodiments, an outer perimeter 1554 of the distal tip 1436 is configured to be joined to the inner surface of the distal end of the return tube 326. In various embodiments, the inner surface of the return tube 326 is metal. Depending on the material of the innermost layer of the return tube, the distal tip 1436 can be joined to the return tube 326 by any suitable means such as metal-to-metal joining processes, adhesives, compressive elements, or the like.

In various embodiments, the outer diameter of the distal tip 1436 is configured to be substantially equal to the inner diameter of the return tube 326. In such an embodiment, bonding the distal tip 1436 to the inner surface of the return tube 326 is configured to center the guidewire lumen 1132 within the shaft 104.

In various embodiments, an exterior surface of the distal tip 1436 includes ridges, barbs, ramps, shoulders, or more than one of these structures which improve the mechanical strength of the bond between the distal tip and the return tube 326 and resist the pressure within the expansion chamber. Examples of such exterior surface structures for distal tip 1436 and structures and techniques for securing the distal tip 1436 to the return tube 326 are shown and described in U.S. Nonprovisional patent application Ser. No. ______, titled “Distal Tip Structure for Cryoablation Probe,” having attorney docket number 115.0438US01, filed on the even date herewith and incorporated herein by reference in its entirety.

The concepts described herein can be applied in the context of and used in connection with cryoablation systems and components described in the following four U.S. nonprovisional patent applications, which are filed on the even date herewith, which are incorporated by reference herein in their entireties: U.S. Nonprovisional patent application Ser. No.______, titled “Cryoablation Catheter Shaft Construction,” having attorney docket number 115.0421USU1; U.S. Nonprovisional patent application Ser. No., ______, titled “Safety Devices for Cryoablation Probe,” having attorney docket number 115.0422USU1; U.S. Nonprovisional patent application Ser. No.______, titled “Multiple Gas Circuit Connector and Method for Cryoablation System,” having attorney docket number 115.0423USU1; and U.S. Nonprovisional patent application Ser. No.______, titled “Distal Tip Structure for Cryoablation Probe,” having attorney docket number 115.0438US01.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cryoablation shaft comprising:
a working fluid circuit;
a vacuum circuit;
an insulated portion, wherein the vacuum circuit runs through the insulated portion; and
an expansion chamber extending distally to the insulated portion;
a guidewire lumen extending through a length of the cryoablation shaft, the guidewire lumen configured for insertion of a guidewire, the guidewire lumen comprising:
a metallic tube; and
a polymer sleeve configured to surround at least a portion of the metallic tube;
a supply tube, the supply tube having a distal outlet in the expansion chamber, wherein fluid from the working fluid circuit travels distally down the cryoablation shaft through supply tube and expands in the expansion chamber;
a return tube surrounding the supply tube; and
an insulating shaft surrounding the return tube.

2. The cryoablation shaft of claim 1, wherein the guidewire lumen is concentrically located within the supply tube, wherein the supply tube is concentrically located within the return tube, wherein at least a portion of the return tube is concentrically located within the insulating shaft.

3. The cryoablation shaft of claim 2, wherein fluid from the working fluid circuit travels distally down the cryoablation shaft through an annular space defined between the return tube and the guidewire lumen.

4. The cryoablation shaft of claim 1, the metallic tube comprising any of nitinol and stainless steel.

5. The cryoablation shaft of claim 1, the polymer sleeve comprising any of polyether block amide and polyethylene terephthalate.

6. The cryoablation shaft of claim 1, wherein at least a portion of the metallic tube comprises slots, and wherein the polymer sleeve is configured to form a seal around the portion of the metallic tube comprising the slots.

7. The cryoablation shaft of claim 1, further comprising a distal tip configured to seal a distal end of the expansion chamber at a distal tip of the cryoablation shaft, wherein the distal tip comprises metal.

8. The cryoablation shaft of claim 7, wherein a distal end of the metallic tube is joined to a proximal end of the distal tip using a metal-to-metal joining process.

9. The cryoablation shaft of claim 7, wherein the distal tip defines a central channel, wherein the central channel is configured to receive the guidewire, wherein a proximal end of the central channel of the distal tip is joined to a distal end of the metallic tube.

10. The cryoablation shaft of claim 1, further comprising a first compressive wrap configured to seal the polymer sleeve to the metallic tube at a distal end of the polymer sleeve.

11. The cryoablation shaft of claim 10, further comprising a second compressive wrap configured to seal the polymer sleeve to the metallic tube at a proximal end of the polymer sleeve.

12. The cryoablation shaft of claim 1, wherein the guidewire lumen may form a curve having a smallest radius of curvature of less than or equal to 30 mm.

13. A guidewire lumen for a cryoablation shaft comprising:
a metallic tube, wherein at least a portion of the metallic tube comprises slots;
a polymer sleeve wherein the polymer sleeve is configured to form a seal around the portion of the metallic tube comprising the slots; and
a distal tip; and
wherein the guidewire lumen is configured to extend through a length of the cryoablation shaft;
wherein the guidewire lumen is configured for insertion of a guidewire; and
wherein the metallic tube is configured to be joined to the distal tip at a distal end of the cryoablation shaft using a metal to metal joining processes.

14. The guidewire lumen of claim 13, wherein the guidewire lumen is concentrically located within a supply tube of the cryoablation shaft, wherein the supply tube is concentrically located within a return tube of the cryoablation shaft, wherein at least a portion of the return tube is concentrically located within an insulating shaft of the cryoablation shaft.

15. The guidewire lumen of claim 13, the metallic tube comprising any of nitinol and stainless steel.

16. The guidewire lumen of claim 13, the polymer sleeve comprising any of polyether block amide and polyethylene terephthalate.

17. The guidewire lumen of claim 13, further comprising a compressive wrap configured to seal the polymer sleeve to the metallic tube.

18. The guidewire lumen of claim 13, wherein the guidewire lumen may form a curve having a smallest radius of curvature of less than or equal to 30 mm.

19. A cryoablation shaft comprising:
a working fluid circuit;
a vacuum circuit;
an insulated portion, wherein the vacuum circuit runs through the insulated portion; and
an expansion chamber;
a guidewire lumen extending through a length of the cryoablation shaft, the guidewire lumen configured for insertion of a guidewire, the guidewire lumen comprising:
a metallic tube wherein at least a portion of the metallic tube comprises slots; and
a polymer sleeve configured to surround at least a portion of the metallic tube, wherein the polymer sleeve is configured to form a seal around the portion of the metallic tube comprising the slots;
a supply tube, the supply tube having a distal outlet in the expansion chamber, wherein fluid from the working fluid circuit travels distally down the cryoablation shaft through supply tube and expands in the expansion chamber;
a return tube surrounding the supply tube; and
an insulating shaft surrounding the return tube; wherein the guidewire lumen is concentrically located within the supply tube, wherein the supply tube is concentrically located within the return tube, wherein the return tube is concentrically located within the insulating shaft.

20. The cryoablation shaft of claim 19, further comprising a distal tip configured to seal a distal end of the expansion chamber at a distal tip of the cryoablation shaft, wherein the distal tip comprises metal.
